(19) United States
(12) Patent Application Publication
(10) Pub. No.: US 2021/0349099 A1
(43) Pub. Date: Nov. 11, 2021

(54) CANCER BIOMARKERS AND METHODS OF USE THEREOF

(71) Applicant: X4 Pharmaceuticals, Inc., Cambridge, MA (US)

(72) Inventors: Yan Wang, Cambridge, MA (US); Simon Paul Fricker, North Grafton, MA (US); Kam Marie Sprott, Needham, MA (US); Jean S. Campbell, Seattle, WA (US); Robert H. Pierce, Seattle, WA (US)

(21) Appl. No.: 16/761,848

(22) PCT Filed: Nov. 6, 2018
(86) PCT No.: PCT/US2018/059482

§ 371 Date: May 6, 2020

Related U.S. Application Data
(60) Provisional application No. 62/582,877, filed on Nov. 7, 2017.
(60) Provisional application No. 62/657,406, filed on Apr. 13, 2018.

(51) Int. Cl. G01N 33/574; A61K 31/4709; A61K 39/395; A61P 35/04

(52) U.S. Cl. CPC G01N 33/57492; A61K 31/4709; A61K 2039/545; A61P 35/04; A61K 39/3955

(57) ABSTRACT

The present invention relates, in part, to certain cancer biomarkers and use thereof in methods for treating cancer, such as in evaluating and/or predicting patient responses to treatment with a CXCR4 inhibitor optionally in combination with a immunotherapeutic agent, in patients with a cancer such as melanoma, including resectable and unresectable melanoma. The present invention also provides a biomarker expression platform, which is a combination of a set of genes or biomarkers that are correlated with response to a CXCR4 inhibitor in a tumor as well as a normalization gene set. A method and system of using the biomarker expression platform to derive biomarker signatures of anti-tumor response and to test patient samples for predictive biomarker signatures are also disclosed.

### CROSS-REFERENCE TO RELATED APPLICATIONS

This application claims the benefit of U.S. Provisional Patent Application Nos. 62/582,877, filed on Nov. 7, 2017; and 62/657,406, filed on Apr. 13, 2018; the entirety of each of which is hereby incorporated by reference.

### FIELD OF THE INVENTION

The present invention relates generally to treatment of cancer using a CXCR4 inhibitor, alone or in combination with an immunotherapeutic agent. More specifically, the present invention relates, in part, to certain cancer biomarkers and their use in methods for treating cancer, for example, in evaluating and/or predicting patient responses to treatment.

### BACKGROUND OF THE INVENTION

The American Cancer Society's estimates for melanoma in the United States for 2017 are: about 87,110 new melanomas will be diagnosed (about 52,170 in men and 34,940 in women). About 9,730 people are expected to die of melanoma. The rates of melanoma have been rising for the last 30 years. When discovered early, melanoma is highly curable with 10-year overall survival rates approaching 95% for stage I melanoma and 45-77% for stage II melanoma after complete surgical resection of the primary melanoma. However, surgical treatment may not be feasible for all patients with advanced melanoma. Patients with unresectable or metastatic disease receive systemic treatment, including immunotherapy (e.g. checkpoint inhibitors (CPI) such as anti-PD-1 and anti-CTLA-4 antibodies) and targeted therapy (e.g. BRAF and/or MEK inhibitors for patients with known genetic mutations). Both checkpoint inhibitor immunotherapy and targeted therapy prolong progression-free survival and overall survival.

Moreover, 30% of patients who have undergone complete resection of their primary melanoma will develop local, in-transit and/or nodal recurrence of their disease. In addition, 10% of melanoma patients present with nodal metastases. Among these stage III patients, complete surgical removal is the main treatment for those with resectable disease; however, the risk of recurrence after surgery is very high. Adjuvant therapies with immunomodulating drugs such as high dose interferon-α and the anti-CTLA-4 antibody ipilimumab have shown to improve the recurrence-free survival in patients with resectable stage III melanoma. The impact of these adjuvant treatments on overall survival is not established.

The benefit of neoadjuvant chemo- and immunotherapy has been demonstrated in several operable cancers. However, tumor development of resistance over time, e.g. via angiogenic escape, is frequently observed and limits the effectiveness of these therapies.

Investigation of CXCR4 inhibitors for use in treating a number of cancers is also warranted. CXCR4 was initially discovered for its involvement in HIV entry and leukocyte trafficking. It is also overexpressed in more than 23 human cancers. CXCR4 is frequently expressed on melanoma cells, particularly the CD133⁺ population that is considered to represent melanoma stem cells; in vitro experiments and murine models have demonstrated that CXCL12, the ligand for CXCR4, is chemotactic for such cells. These data underscore the significant, unmet need for study of CXCR4 inhibitors to treat cellular proliferative disorders that result from overexpression or aberrant expression of CXCR4.

### BRIEF DESCRIPTION OF THE DRAWINGS

FIG. 1 shows photographs of a metastatic melanoma human tumor sample stained with CD8⁺ single-marker IHC stain demonstrating a large increase in CD8⁺ T cells at the tumor margin after dosing with a combination of X4P-001 and pembrolizumab.

FIG. 2 shows representative granzyme B IHC staining at baseline (FIG. 2, panel A) and following 21 days of X4P-001 treatment (FIG. 2, panel B). FIG. 2, panel C shows the fold change of granzyme B positivity post-treatment for all evaluable samples. Quantification was performed using HALO™ software and the entire tumor area was scored. FIG. 2, panel D shows the granzyme B RNA expression level for 5 patients with both pre- and post-X4P-001 single agent treatment evaluable biopsies. The RNA expression data in panel D was obtained using NanoString as described herein.

FIG. 3A shows gene expression scores pre- and post-dosing with X4P-001 for the cytotoxic T lymphocyte (CTL) gene signature. Gene scores were calculated for each patient sample from the geometric mean of normalized counts for CD8A, CD8B, FLTLG, GZAM, and PRF1. The mean was Log 10-transformed to generate the Gene Expression score. The gene expression score increased for each one of the five patients. FIG. 3B shows CTL gene signature data for patients in FIG. 3A as well as additional patients. In the NanoString analyses here and in later FIGS. 6A, 6B and FIGS. 17A and 17B, each set of data is normalized based on a set of housekeeping genes in each individual run of the experiment. Integration of data sets from all patients using the NanoString algorithm results in an integrated value based upon normalization from the combined data set, which can differ from the apparent value from each individual data set.

FIG. 4 shows the results of IHC CD8 staining for patient #5 pre- and post-dosing with X4P-001. CD8 expression was visibly increased after dosing. CD8⁺ T cells density in tumor microenvironment was increased from 1045 per square millimeter to 1370 per square millimeter.

FIG. 5 shows a bar graph of mIF results for melanoma patient #5 demonstrating that treatment with X4P-001 increased the percentage of CD4, CD8, PD-1, and PDL-1 positive cells in the TME. The percentages of T_{reg} (FoxP3⁺) cells and macrophages (CD68⁺/CD163⁺; 24.1% vs. 25.4%; not shown) were not altered.

FIG. 6A shows gene expression scores pre- and post-dosing with X4P-001 for the interferon gamma (IFN-γ) gene signature. Gene scores were calculated for each patient sample from the geometric mean of normalized counts for IFN-gamma, CXCL9, CXCL10, HLA-DRA, IDO1, and STAT1. The mean was Log 10-transformed to generate the Gene Expression score. The Gene Expression Score increased for each one of the five patients. FIG. 6B shows gene expression scores pre- and post-dosing with X4P-001 for the IFN-γ gene signature in additional patients.

FIG. 7 shows signal quantification of single marker immunohistochemistry (IHC) data for biomarkers CD8⁺, CD3⁺, and FoxP3 obtained by HALO. EOT=End Of Treatment (three week treatment of X4P-001+6 weeks of combination of X4P-001 with pembrolizumab).

FIG. 8 shows the dosage schedule for a nine (9) week study of X4P-001 monotherapy and in combination with pembrolizumab.

FIG. 9A shows representative CD8 and FoxP3 staining of biopsy samples under low magnification (Panel A) and high magnification (Panel B) following X4P-001 monotherapy. FIG. 9B shows images of formalin-fixed paraffin-embedded melanoma samples. The samples were stained sequentially with a 6-component immunophenotyping antibody panel, including CD4, CD8, PD-1, PD-L1, macrophage cocktail (CD68+CD163), and FoxP3. DAPI was used as a nuclear counterstain. Antibodies were detected using HRP-catalyzed deposition of fluorescent tyramide substrates (Opal, Perkin-Elmer). Images were obtained using spectral imaging, autofluorescence subtraction and unmixing (Vectra 3.0, Perkin-Elmer), and analyzed using HALO™ image analysis software.

FIG. 10 shows a line graph of mIF results for melanoma patients 2, 3, 5, 8, and 9 demonstrating an increase in CD8 cells relative to Treg cells following X4P-001 monotherapy.

FIG. 11 shows representative CD8, Ki-67, and melanoma cell staining under low power scan of an entire biopsy from patient 5 (Panel 1a) and under unmixed high-power imaging of the melanoma invasive front (Panel 1b).

FIG. 12 shows a bar graph of CD8⁺ T cell and proliferating CD8⁺ T cell (Ki-67⁺) densities across an entire biopsy sample from patient 5. The left Y axis is CD8⁺Ki67⁺ cells (# cells/mm²); the right Y axis is CD+ Tc cells (# cells/mm²). In FIGS. 13, 14 and 15, the images represent the graphical output from the nearest neighbor analysis module, with unlabeled cells rendered as gray. After X4P-001 monotherapy, proliferative CD8⁺ T cells surround and infiltrate the tumor lesion. The average distance between CD8⁺ cells and the nearest tumor cell decreases from 95 microns at baseline to 43 microns after X4P-001 monotherapy, and the number of unique neighbors increases, indicating enhanced infiltration.

FIG. 13 shows the distance measurements between CD8⁺ T cells and their nearest melanoma cell neighbors on Day 1 (pre-treatment).

FIG. 14 shows the distance measurements between CD8⁺ T cells and their nearest melanoma cell neighbors on after 4 weeks of monotherapy with X4P-001.

FIG. 15 shows the distance measurements between CD8⁺ T cells and their nearest melanoma cell neighbors on after end of treatment.

FIG. 16 shows gene expression scores pre- and post-dosing with X4P-001 for the Antigen Presentation/Processing gene signature. Gene scores were calculated for each patient sample from the geometric mean of normalized counts for B2M, CD74, CTSL, CTSS, HLA-DMA, HLA-DMB, HLA-DOB, HLA-DPA1, HLA-DPB1, HLA-DQA1, HLA-DQB1, HLA-DRA, HLA-DRB1, HLA-DRB3, PSMB8, PSMB9, TAP1, and TAP2. The mean was Log 10-transformed to generate the Gene Expression score. The Gene Expression Score increased for each one of the five patients.

FIG. 17A shows gene expression scores pre- and post-dosing with X4P-001 for the Tumor Inflammation gene signature. Gene scores were calculated for each patient sample from the geometric mean of normalized counts for CCL5, CD27, CD274, CD276, CD8A, CMKLR1, CXCL9, CXCR6, HLA-DQA1, HLA-DRB1, HLA-E, IDO1, LAG3, NKG7, PDCD1LG2, PSMB10, STAT1, and TIGIT The mean was Log 10-transformed to generate the Gene Expression score. The Gene Expression Score increased for each one of the five patients.

FIG. 17B shows gene expression scores pre- and post-dosing with X4P-001 for the Tumor Inflammation gene signature in the patients in FIG. 17A with data for additional patients included.

FIG. 18, FIG. 19, and FIG. 20 show B16-OVA tumor growth in C57BL/6 mice over sixteen (16) days with treatment with control, X4P-136, anti-PD-L1, anti-PD-L1+X4P-136, anti-PD-1, anti PD-1+X4P-136, anti-CLTA-4+anti-PD-L1, and anti-CTLA-4+anti-PD-L1+X4P-136.

FIG. 21 shows representative dissections of mice implanted with B16-OVA tumors after sixteen (16) days of treatment.

FIG. 22 shows a bar graph depicting the difference in peripheral white blood cells at baseline and two (2) hours post X4P-136 injection.

FIG. 23 shows bar graphs depicting changes in immune cell phenotype in the tumor microenvironment following treatment with the captioned therapies.

FIG. 24 shows a Western blot depicting the effect of indicated treatments on HIF-2α expression and Akt activity.

FIG. 25 shows a Western blot depicting the effect of indicated treatments on p21 and p27 induction and Cyclin D1 expression.

FIG. 26 shows a bar graph depicting the dose response effect of X4P-136 on transcription via HIF-2a response elements under normoxic and hypoxic conditions.

FIG. 27 shows a bar graph depicting the dose response effect of X4P-136 on in vitro tumor cell invasion under normoxic and hypoxic conditions.

FIG. 28 and FIG. 29 show multiplex IHC and HALO image data demonstrating that X4P-001 monotherapy increases CD8+ cell density at the tumor interface in melanoma patients. CD8-labeled cells within 100 μM of the inside or outside of the tumor boundary with normal tissue were counted. The number of CD8+ cells/mm² was plotted against distance from the boundary in 25 μM bands. After 3 weeks of X4P-001 monotherapy, the total density of CD8+ cells within the boundary area was increased four-fold compared with baseline.

FIG. 30 shows mIF data demonstrating immune cell alterations following single agent treatment (X4P-001). Biopsy samples were obtained at baseline (top row) and at the end of X4P-001 monotherapy (bottom row). The left column shows biopsy samples with outlines of normal tissue (outer line) and the tumor border (inner line). The center column shows the enlarged boxed regions from the left column stained with the markers CD163, CD206, VISTA, COX-2, CD3, B7H3, and DAPI. The right column contains higher magnification views of the boxed regions in the center panel. X4P-001 leads to increased numbers of CD3+ cells within tumor borders and decreased expression of VISTA, a check point molecule that inhibits T cell activation and proliferation.

### DETAILED DESCRIPTION OF CERTAIN EMBODIMENTS OF THE INVENTION

General Description of Certain Embodiments of the Invention

Diagnosis, prognosis, and treatment of cancer is greatly aided by the identification of intratumoral expression patterns for sets of genes, changes in levels of immune-related cells in the tumor microenvironment, or other changes in the tumor microenvironment, referred to herein generally as “biomarkers” or more specifically in relation to gene expression patterns as “gene signatures,” “gene expression biomarkers,” or “molecular signatures,” which are characteristic of particular types or subtypes of cancer, and which are associated with clinical outcomes. Such biomarkers may be associated with clinical outcomes. If such an association is predictive of a clinical response, the biomarker is advantageously used in methods of selecting or stratifying patients as more (or less, as the case may be) likely to benefit from a treatment regimen, such as one of those disclosed herein. Tumor samples with biomarkers that are predictive of a positive response to treatment are referred to herein as “biomarker positive” or “biomarker high.” Conversely, tumor samples with biomarker profiles that are not predictive of a positive response are referred to herein as “biomarker negative” or “biomarker low.” Alternative terms can be used depending upon the biomarker, but a higher amount, or “biomarker high” usually can be described using alternative terminology, such as “biomarker positive” or “biomarker+” while a lower amount of a biomarker or “biomarker low” usually can be described using alternative terminology, such as “biomarker negative” or “biomarker −.”

It has now been surprisingly found that levels of CD8⁺ T cells (or CD8⁺ T cells/T_{reg} ratio); CD8⁺Ki-67⁺ T cells; granzyme B; an IFN-γ signature score; a CTL signature score; an antigen presentation/processing signature score; a tumor inflammation signature score; a VISTA biomarker panel; and/or PD-L1 expression are useful as biomarkers in a method described herein, such as a method of treating or diagnosing a cancer such as metastatic melanoma.

Accordingly, in one aspect, the present invention provides a method of identifying a patient with a cancerous tumor who will benefit from treatment with a CXCR4 inhibitor, comprising:
(a) obtaining a first tumor sample prior to administration of the CXCR4 inhibitor to the patient;
(b) measuring a level in the first tumor sample of one or more biomarkers selected from CD8⁺ T cells (or CD8⁺ T cells/T_{reg} ratio), CD8⁺Ki-67⁺ T cells, granzyme B, an IFN-γ signature score, a CTL signature score, an antigen presentation/processing signature score, a tumor inflammation signature score, a VISTA biomarker panel, or PD-L1 expression;
(c) administering to the patient an effective amount of a CXCR4 inhibitor and optionally an immunotherapeutic agent;
(d) obtaining a second tumor sample after administration of the CXCR4 inhibitor to the patient; and
(e) measuring a level in the second tumor sample of one or more biomarkers selected from CD8⁺ T cells (or CD8⁺ T cells/T_{reg} ratio), CD8⁺Ki-67⁺ T cells, granzyme B, an IFN-γ signature score, a CTL signature score, an antigen presentation/processing signature score, a tumor inflammation signature score, a VISTA biomarker panel, or PD-L1 expression.

In another aspect, the present invention provides a method of identifying a patient with a cancer who is likely to benefit, or has an increased probability of benefitting relative to an otherwise similar patient, from treatment with a CXCR4 inhibitor optionally in combination with an immunotherapeutic agent, comprising:

(a) obtaining a first tumor sample prior to administration of the CXCR4 inhibitor to the patient;

(b) measuring a level in the first tumor sample of one or more biomarkers selected from CD8⁺ T cells (or CD8⁺ T cells/T_{reg} ratio), CD8⁺Ki-67⁺ T cells, granzyme B, an IFN-γ signature score, a CTL signature score, an antigen presentation/processing signature score, a tumor inflammation signature score, a VISTA biomarker panel, or PD-L1 expression;

(c) administering to the patient an effective amount of a CXCR4 inhibitor and optionally an immunotherapeutic agent;

(d) obtaining a second tumor sample after administration of the CXCR4 inhibitor to the patient; and

(e) measuring a level in the second tumor sample of one or more biomarkers selected from CD8⁺ T cells (or CD8⁺ T cells/T_{reg} ratio), CD8⁺Ki-67⁺ T cells, granzyme B, an IFN-γ signature score, a CTL signature score, an antigen presentation/processing signature score, a tumor inflammation signature score, a VISTA biomarker panel, or PD-L1 expression; wherein the cancer response to step (c) is predictive of the likelihood of successful treatment of the cancer based on a greater or lesser response of the cancer compared with one or more similar patients and as evaluated using one or more of the biomarkers.

In some embodiments, the first tumor sample and/or second tumor sample are assayed in vitro or ex vivo.

In another aspect, the present invention provides a method of assaying a tumor sample taken from a patient in vitro or ex vivo to determine if a tumor in the patient will respond, or has an increased probability of responding, to treatment with a CXCR4 inhibitor optionally in combination with an immunotherapeutic agent, comprising:
(a) obtaining a first tumor sample prior to administration of the CXCR4 inhibitor to the patient;

(b) measuring a level in the first tumor sample of one or more biomarkers selected from CD8⁺ T cells (or CD8⁺ T cells/T_{reg} ratio), CD8⁺Ki-67⁺ T cells, granzyme B, an IFN-γ signature score, a CTL signature score, an antigen presentation/processing signature score, a tumor inflammation signature score, a VISTA biomarker panel, or PD-L1 expression; and, optionally,

(c) administering to the patient an effective amount of a CXCR4 inhibitor and optionally an immunotherapeutic agent, if the tumor in the patient will respond, or has an increased probability of responding, to treatment with a CXCR4 inhibitor optionally in combination with an immunotherapeutic agent.

In another aspect, the present invention provides a method of treating a cancer, e.g., tumor, in a patient who either does not respond to monotherapy with an immunotherapeutic agent or whose cancer has become refractory after initially responding to monotherapy with an immunotherapeutic agent, comprising:

(a) obtaining a first tumor sample prior to administration of the CXCR4 inhibitor to the patient;

(b) measuring a level in the first tumor sample of one or more biomarkers selected from CD8⁺ T cells or CD8⁺ T cells/T_{reg} ratio, CD8⁺Ki-67⁺ T cells, granzyme B, an IFN-γ signature score, a CTL signature score, an antigen presentation/processing signature score, a tumor inflammation signature score, a VISTA biomarker panel, or PD-L1 expression;

(c) administering to the patient an effective amount of a CXCR4 inhibitor and optionally an immunotherapeutic agent;

(d) obtaining a second tumor sample after administration of the CXCR4 inhibitor to the patient; and

(e) measuring a level in the second tumor sample of one or more biomarkers selected from CD8⁺ T cells or CD8⁺ T cells/T_{reg} ratio, CD8⁺Ki-67⁺ T cells, granzyme B, an IFN-γ signature score, a CTL signature score, an antigen presentation/processing signature score, a tumor inflammation signature score, a VISTA biomarker panel, or PD-L1 expression;

wherein the tumor response to step (c) is predictive of the likelihood of successful treatment of the tumor based on a greater or lesser response of the tumor compared with one or more similar patients and as evaluated using one or more of the biomarkers.

In some embodiments, the present invention provides a method of predicting whether a cancer, e.g., tumor, will respond to treatment with an immunotherapeutic agent after treatment with a CXCR4 inhibitor, comprising:

(a) obtaining a first tumor sample prior to administration of the CXCR4 inhibitor to the patient;

(b) measuring a level in the first tumor sample of one or more biomarkers selected from CD8⁺ T cells or CD8⁺ T cells/T_{reg} ratio, CD8⁺Ki-67⁺ T cells, granzyme B, an IFN-γ signature score, a CTL signature score, an antigen presentation/processing signature score, a tumor inflammation signature score, a VISTA biomarker panel, or PD-L1 expression;

(c) administering to the patient an effective amount of a CXCR4 inhibitor and optionally an immunotherapeutic agent;

(d) obtaining a second tumor sample after administration of the CXCR4 inhibitor to the patient; and

(e) measuring a level in the second tumor sample of one or more biomarkers selected from CD8⁺ T cells or CD8⁺ T cells/T_{reg} ratio, CD8⁺Ki-67⁺ T cells, granzyme B, an IFN-γ signature score, a CTL signature score, an antigen presentation/processing signature score, a tumor inflammation signature score, a VISTA biomarker panel, or PD-L1 expression;

wherein the tumor response to step (c) is predictive of the likelihood of successful treatment of the tumor based on a greater or lesser response of the tumor compared with one or more similar patients and as evaluated using one or more of the biomarkers.

In some embodiments, treatment with a CXCR4 inhibitor primes the tumor microenvironment such that the tumor becomes more likely to respond to an immunotherapeutic agent. In some embodiments, the tumor does not respond to monotherapy with a PD-1 inhibitor, but becomes primed and responds to the PD-1 inhibitor when combined with a CXCR4 inhibitor. In some embodiments, the tumor initially responds to the PD-1 inhibitor or another checkpoint inhibitor, but becomes refractory. In some embodiments, after treatment with a CXCR4 inhibitor, the tumor can be treated effectively with the PD-1 inhibitor or other immunotherapeutic agent.

In some embodiments, the CXCR4 inhibitor is administered in combination with an immunotherapeutic agent. In some embodiments, the CXCR4 inhibitor is X4P-001 or X4-136, or pharmaceutically acceptable salts thereof. In some embodiments, the CXCR4 inhibitor is X4P-001 or a pharmaceutically acceptable salt thereof. In some embodiments, the CXCR4 inhibitor is X4-136 or a pharmaceutically acceptable salt thereof. X4P-001 has the structure depicted below:

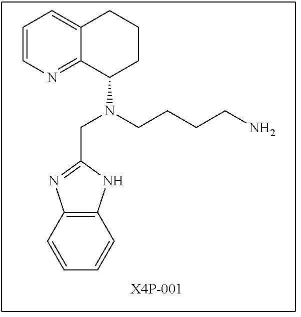 [SMILES: NCCCCN(Cc1nc2ccccc2[nH]1)[C@H]1CCCc2cccnc21]

X4P-001 and the synthesis thereof is described in detail in U.S. Pat. No. 7,354,934, which is hereby incorporated by reference.

X4-136 has the structure depicted below:

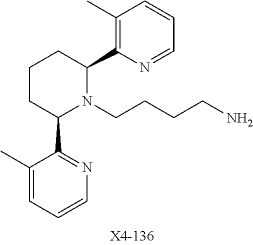 [SMILES: Cc1cccnc1[C@H]1CCC[C@@H](c2ncccc2C)N1CCCCN]

X4-136 and the synthesis thereof is described in detail in U.S. Pat. No. 7,550,484.

In some embodiments, the immunotherapeutic agent is a checkpoint inhibitor. In some embodiments, the checkpoint inhibitor is a PD-1 antagonist. In some embodiments, the PD-1 antagonist is selected from nivolumab, pembrolizumab, a pembrolizumab biosimilar, or a pembrolizumab variant. In some embodiments, the checkpoint inhibitor is pembrolizumab.

In some embodiments, the cancerous tumor is a solid tumor. In some embodiments, the solid tumor is melanoma. In some embodiments, the melanoma is malignant melanoma, advanced melanoma, metastatic melanoma, or Stage I, II, III, or IV melanoma. In some embodiments, the melanoma is resectable. In some embodiments, the melanoma is unresectable. In some embodiments, the melanoma is unresectable advanced or unresectable metastatic melanoma. In some embodiments, the patient has not previously undergone treatment with an immune checkpoint inhibitor such as anti-CTLA-4, PD-1, or PD-L1, or previously undergone oncolytic virus therapy.

In some embodiments, the above method is useful in the identification of a patient who will benefit from treatment with a CXCR4 inhibitor optionally in combination with an immunotherapeutic agent. Such a patient is characterized in that the level of one or more biomarkers selected from CD8⁺ T cells or CD8⁺ T cells/T_{reg} ratio, CD8⁺Ki-67⁺ T cells, granzyme B, an IFN-γ signature score, a CTL signature score, an antigen presentation/processing signature score, a tumor inflammation signature score, a VISTA biomarker panel, or PD-L1 expression is higher in the second tumor sample than in the first tumor sample. In some embodiments, when the level of one or more biomarkers selected from CD8⁺ T cells or CD8⁺ T cells/T_{reg} ratio, CD8⁺Ki-67⁺ T cells, granzyme B, an IFN-γ signature score, a CTL signature score, an antigen presentation/processing signature score, a tumor inflammation signature score, a VISTA biomarker panel, or PD-L1 expression is higher in the second tumor sample than in the first tumor sample, then the patient is administered one or more additional doses of the CXCR4 inhibitor. This is because such a patient is considered likely to benefit from continued treatment with the CXCR4 inhibitor and, optionally, the immunotherapeutic agent.

In some embodiments, the first tumor sample and/or second tumor sample are assayed in vitro or ex vivo.

In another aspect, the present invention provides a method of treating a cancer with a CXCR4 inhibitor, comprising
(a) obtaining a first tumor sample prior to administration of the CXCR4 inhibitor to the patient;
(b) measuring a level in the first tumor sample of one or more biomarkers selected from CD8⁺ T cells or CD8⁺ T cells/T_{reg} ratio, CD8⁺Ki-67⁺ T cells, granzyme B, an IFN-γ signature score, a CTL signature score, an antigen presentation/processing signature score, a tumor inflammation signature score, a VISTA biomarker panel, or PD-L1 expression;
(c) administering to the patient an effective amount of a CXCR4 inhibitor and optionally an immunotherapeutic agent;
(d) obtaining a second tumor sample after administration of the CXCR4 inhibitor to the patient; and
(e) measuring a level in the second tumor sample of one or more biomarkers selected from CD8⁺ T cells or CD8⁺ T cells/T_{reg} ratio, CD8⁺Ki-67⁺ T cells, granzyme B, an IFN-γ signature score, a CTL signature score, an antigen presentation/processing signature score, a tumor inflammation signature score, a VISTA biomarker panel, or PD-L1 expression; wherein:
   when the level of one or more biomarkers selected from CD8⁺ T cells or CD8⁺ T cells/T_{reg} ratio, CD8⁺Ki-67⁺ T cells, granzyme B, an IFN-γ signature score, a CTL signature score, an antigen presentation/processing signature score, a tumor inflammation signature score, a VISTA biomarker panel, or PD-L1 expression is higher in the second tumor sample than in the first tumor sample, then the patient is administered one or more additional doses of the CXCR4 inhibitor and optionally the immunotherapeutic agent.

In another aspect, the present invention provides a method of evaluating a patient response to a CXCR4 inhibitor optionally in combination with an immunotherapeutic agent, comprising the steps of:
(a) obtaining a first tumor sample prior to administration of the CXCR4 inhibitor to the patient;
(b) measuring a level in the first tumor sample of one or more biomarkers selected from CD8⁺ T cells or CD8⁺ T cells/T_{reg} ratio, CD8⁺Ki-67⁺ T cells, granzyme B, an IFN-γ signature score, a CTL signature score, an antigen presentation/processing signature score, a tumor inflammation signature score, a VISTA biomarker panel, or PD-L1 expression;
(c) administering to the patient an effective amount of a CXCR4 inhibitor and optionally an immunotherapeutic agent;
(d) obtaining a second tumor sample after administration of the CXCR4 inhibitor to the patient; and
(e) measuring a level in the second tumor sample of one or more biomarkers selected from CD8⁺ T cells or CD8⁺ T cells/T_{reg} ratio, CD8⁺Ki-67⁺ T cells, granzyme B, an IFN-γ signature score, a CTL signature score, an antigen presentation/processing signature score, a tumor inflammation signature score, a VISTA biomarker panel, or PD-L1 expression;
   wherein the tumor response to step (c) is evaluated to split, classify, or stratify the patient into one of two or more groups based on a greater or lesser response of the tumor compared with one or more similar patients.

In another aspect, the present invention provides a method of predicting a patient response to a CXCR4 inhibitor optionally in combination with an immunotherapeutic agent, comprising the steps of:
(a) obtaining a first tumor sample prior to administration of the CXCR4 inhibitor to the patient;
(b) measuring a level in the first tumor sample of one or more biomarkers selected from CD8⁺ T cells or CD8⁺ T cells/T_{reg} ratio, CD8⁺Ki-67⁺ T cells, granzyme B, an IFN-γ signature score, a CTL signature score, an antigen presentation/processing signature score, a tumor inflammation signature score, a VISTA biomarker panel, or PD-L1 expression;
(c) administering to the patient an effective amount of a CXCR4 inhibitor and optionally an immunotherapeutic agent;
(d) obtaining a second tumor sample after administration of the CXCR4 inhibitor to the patient; and
(e) measuring a level in the second tumor sample of one or more biomarkers selected from CD8⁺ T cells or CD8⁺ T cells/T_{reg} ratio, CD8⁺Ki-67⁺ T cells, granzyme B, an IFN-γ signature score, a CTL signature score, an antigen presentation/processing signature score, a tumor inflammation signature score, a VISTA biomarker panel, or PD-L1 expression;
   wherein the tumor response to step (c) is predictive of the likelihood of successful treatment of the tumor based on a greater or lesser response of the tumor compared with one or more similar patients and as evaluated using one or more of the biomarkers.

In another aspect, the present invention provides a method of predicting a treatment response of a cancer in a patient to a CXCR4 inhibitor optionally in combination with an immunotherapeutic agent, comprising the steps of:
(a) obtaining a tumor sample prior to administration of the CXCR4 inhibitor to the patient;
(b) measuring a level in the tumor sample of one or more biomarkers selected from CD8⁺ T cells or CD8⁺ T cells/T_{reg} ratio, CD8⁺Ki-67⁺ T cells, granzyme B, an IFN-γ signature score, a CTL signature score, an antigen presentation/processing signature score, a tumor inflammation signature score, a VISTA biomarker panel, or PD-L1 expression; (c) treating the tumor sample or a reference sample;
(e) measuring a level in the treated tumor or reference sample of one or more biomarkers selected from CD8⁺ T cells or CD8⁺ T cells/T_{reg} ratio, CD8⁺Ki-67⁺ T cells, granzyme B, an IFN-γ signature score, a CTL signature score, an antigen presentation/processing signature score, a tumor inflammation signature score, a VISTA biomarker panel, or PD-L1 expression;
(f) comparing one of more biomarkers in the pre-treatment tumor sample with one or more biomarkers in the treated serum sample or treated reference sample; and
(g) optionally, proceeding with administration of the CXCR4 inhibitor to the patient, optionally in combination with an immunotherapeutic agent, if such administration is predicted to have an equivalent or higher likelihood of success relative to an alternative method of treating the cancer;
   wherein the biomarker change in response to step (c) is predictive of the likelihood of successful treatment of the cancer based on a greater or lesser biomarker change compared with one or more similar patients and as evaluated using one or more of the biomarkers.

In some embodiments, the reference sample is from another patient, such as a patient with a similar cancer; or the reference sample may be a culture or other in vitro sample of a similar cancer.

In some embodiments, the first tumor sample and/or second tumor sample are assayed in vitro or ex vivo.

In another aspect, the present invention provides a method of monitoring a patient response to a CXCR4 inhibitor optionally in combination with an immunotherapeutic agent, comprising the steps of:
(a) obtaining a first tumor sample prior to administration of the CXCR4 inhibitor to the patient;
(b) measuring a level in the first tumor sample of one or more biomarkers selected from CD8⁺ T cells or CD8⁺ T cells/T_{reg} ratio, CD8⁺Ki-67⁺ T cells, granzyme B, an IFN-γ signature score, a CTL signature score, an antigen presentation/processing signature score, a tumor inflammation signature score, a VISTA biomarker panel, or PD-L1 expression;
(c) administering to the patient an effective amount of a CXCR4 inhibitor and optionally an immunotherapeutic agent;
(d) obtaining a one or more subsequent tumor samples after administration of the CXCR4 inhibitor to the patient; and
(e) measuring a level in the subsequent tumor sample(s) of one or more biomarkers selected from CD8⁺ T cells or CD8⁺ T cells/T_{reg} ratio, CD8⁺Ki-67⁺ T cells, granzyme B, an IFN-γ signature score, a CTL signature score, an antigen presentation/processing signature score, a tumor inflammation signature score, a VISTA biomarker panel, or PD-L1 expression;
   wherein the levels of one of more biomarkers in the first tumor sample and subsequent tumor samples can be compared and changes in one or more of the biomarkers indicate a patient response.

In some embodiments, the patient response to a CXCR4 inhibitor optionally in combination with an immunotherapeutic agent is measured once per week or every two weeks. In some embodiments, the patient response is measured once a month. In some embodiments, the patient's response is measured bimonthly. In some embodiments, the patient's response is measured quarterly (once every three months). In some embodiments, the patient's response is measured annually.

In some embodiments, the patient response to a CXCR4 inhibitor optionally in combination with an immunotherapeutic agent is monitored while undergoing treatment. In some embodiments, the patient response is monitored after treatment is concluded.

In another aspect, the present invention provides a method of deriving a biomarker signature that is predictive of an antitumor response to treatment with a CXCR4 inhibitor optionally in combination with a PD-1 antagonist for a tumor, comprising:
(a) obtaining a pre-treatment tumor sample from each patient in a patient cohort diagnosed with the tumor type;
(b) obtaining, for each patient in the cohort, an anti-tumor response value following treatment with the CXCR4 inhibitor optionally in combination with the PD-1 antagonist;
(c) measuring the raw biomarker levels in each tumor sample for each gene in a biomarker platform, wherein the biomarker platform comprises a clinical response biomarker set of CD8⁺ T cells or CD8⁺ T cells/T_{reg} ratio, CD8⁺Ki-67⁺ T cells, granzyme B, an IFN-γ signature score, a CTL signature score, an antigen presentation/processing signature score, a tumor inflammation signature score, a VISTA biomarker panel, or PD-L1 expression;
(d) normalizing, for each tumor sample, each of the measured raw biomarker levels for the clinical response biomarkers using the measured biomarker levels of a set of normalization biomarkers; and
(e) comparing the biomarker levels for all of the tumor samples and the antitumor response values for all of the patients in the cohort to select a cutoff for the biomarker signature score that divides the patient cohort to meet a target biomarker clinical utility criterion.

In some embodiments, the biomarker platform comprises a gene expression platform that comprises a clinical response gene set. In some embodiments, the method further comprises the steps of:
(f) weighting, for each tumor sample and each biomarker, such as a gene in a gene signature of interest, the normalized biomarker (e.g., RNA biomarker) expression levels using a pre-defined multiplication coefficient for that gene;
(g) adding, for each patient, the weighted biomarker (e.g., RNA biomarker) expression levels to generate a biomarker signature score, e.g., a gene signature score, for each patient in the cohort.

In another aspect, the present invention provides a method of testing a sample of a tumor removed from a patient for the presence or absence of a gene signature biomarker of anti-tumor response of the tumor to a CXCR4 inhibitor optionally in combination with a PD-1 antagonist, comprising:
(a) measuring the raw RNA level in the tumor sample for each gene in a gene expression platform, wherein the gene expression platform comprises a clinical response gene set selected from an IFN-γ signature, a CTL signature, an antigen presentation/processing signature, a tumor inflammation signature, CD8A, CD8B, granzyme B gene expression, or PD-L1 expression and a normalization gene set of housekeeping genes, and optionally wherein about 80%, or about 90%, of the clinical response genes exhibit intratumoral RNA levels that are positively correlated with the anti-tumor response;
(b) normalizing the measured raw RNA level for each clinical response gene in a pre-defined gene signature for the tumor sample using the measured RNA levels of the normalization genes, wherein the pre-defined gene signature consists of at least 2 of the clinical response genes, thus obtaining a gene signature score;
(c) comparing the gene signature score to a reference score for the gene signature and tumor; and
(d) classifying the tumor sample as biomarker high or biomarker low;
   wherein if the generated score is equal to or greater than the reference score, then the tumor sample is classified as biomarker high, and if the generated score is less than the reference score, then the tumor sample is classified as biomarker low.

In some embodiments, after step (b) the method comprises the further steps of:
(i) weighting each normalized RNA value using a pre-defined multiplication co-efficient;
(ii) adding the weighted RNA expression levels to generate a weighted gene signature score.

In some embodiments, the normalization gene set comprises about 10 to about 12 housekeeping genes, or about 30-40 housekeeping genes.

In another aspect, the present invention provides a method of testing a sample of a tumor removed from a patient for the presence or absence of a biomarker signature of antitumor response of the tumor to a CXCR4 inhibitor optionally in combination with a PD-1 antagonist, comprising:
(a) measuring the raw biomarker level in the tumor sample for each biomarker in a biomarker platform, wherein the biomarker platform comprises a clinical response biomarker set selected from CD8⁺ T cells or CD8⁺ T cells/T_{reg} ratio, CD8⁺Ki-67⁺ T cells, granzyme B, an IFN-γ signature score, a CTL signature score, an antigen presentation/processing signature score, a tumor inflammation signature score, a VISTA biomarker panel, or PD-L1 expression and a normalization biomarker set, and optionally wherein about 80%, or about 90%, of the clinical response biomarkers exhibit intratumoral biomarker levels that are positively correlated with the anti-tumor response;
(b) normalizing the measured raw biomarker level for each clinical response biomarker in a pre-defined biomarker signature for the tumor sample using the measured biomarker levels of the normalization biomarkers, wherein the pre-defined biomarker signature consists of at least 2 of the clinical response biomarkers;
(c) comparing the normalized biomarker levels and a set of reference biomarker levels for the tumor; and
(d) classifying the tumor sample as biomarker high or biomarker low;
   wherein if the normalized biomarker levels are equal to or greater than the reference biomarker levels, then the tumor sample is classified as biomarker high, and if the normalized biomarker levels are less than the reference biomarker levels, then the tumor sample is classified as biomarker low.

In some embodiments, the normalization biomarker set comprises about 10 to about 12 housekeeping genes, or about 30-40 housekeeping genes. In some embodiments, the level of CD8⁺ T cells is measured by CD8A and/or CD8B expression. In some embodiments, the CD8⁺ T cells/T_{reg} ratio is measured by determining the expression level of FoxP3 compared with CD8A and/or CD8B.

In another aspect, the present invention provides a system for testing a sample of a tumor removed from a patient for the presence or absence of a biomarker signature of anti-tumor response of the tumor to a CXCR4 inhibitor optionally in combination with a PD-1 antagonist, comprising:
(i) a sample analyzer for measuring raw biomarker levels in a biomarker platform, wherein the biomarker platform consists of a set of clinical response biomarkers selected from CD8⁺ T cells or CD8⁺ T cells/T_{reg} ratio, CD8⁺Ki-67⁺ T cells, granzyme B, an IFN-γ signature score, a CTL signature score, an antigen presentation/processing signature score, a tumor inflammation signature score, a VISTA biomarker panel, or PD-L1 expression; and a set of normalization biomarkers; and
(ii) a computer program for receiving and analyzing the measured biomarker levels to:
   (a) normalize the measured raw biomarker level for each clinical response biomarker in a pre-defined biomarker signature for the tumor using the measured levels of the normalization biomarkers;
   (b) compare the generated biomarker level to a reference level for the biomarker signature and tumor; and
   (c) classify the tumor sample as biomarker high or biomarker low, wherein if the generated score is equal to or greater than the reference score, then the tumor sample is classified as biomarker high, and if the generated score is less than the reference score, then the tumor sample is classified as biomarker low.

In another aspect, the present invention provides a system for testing a sample of a tumor removed from a patient for the presence or absence of a biomarker signature of anti-tumor response of the tumor to a CXCR4 inhibitor optionally in combination with a PD-1 antagonist, comprising:
(i) a sample analyzer for measuring raw biomarker levels in a biomarker platform, wherein the biomarker platform consists of a set of clinical response biomarkers selected from CD8⁺ T cells or CD8⁺ T cells/T_{reg} ratio, CD8⁺Ki-67⁺ T cells, granzyme B, an IFN-γ signature score, a CTL signature score, an antigen presentation/processing signature score, a tumor inflammation signature score, a VISTA biomarker panel, or PD-L1 expression; and a set of normalization biomarkers; and
(ii) a computer program for receiving and analyzing the measured biomarker levels to
   (a) normalize the measured raw biomarker level for each clinical response biomarker in a pre-defined biomarker signature for the tumor using the measured levels of the normalization biomarkers;
   (b) weight each normalized biomarker level using a pre-defined multiplication coefficient;
   (c) add the weighted biomarker levels to generate a biomarker signature score; (d) compare the generated score to a reference score for the biomarker signature and tumor; and
   (e) classify the tumor sample as biomarker high or biomarker low, wherein if the generated score is equal to or greater than the reference score, then the tumor sample is classified as biomarker high, and if the generated score is less than the reference score, then the tumor sample is classified as biomarker low.

In some embodiments, the biomarker comprises the RNA expression level of a gene described herein, such as CD8A, CD8B, FoxP3, granzyme B, an IFN-γ signature gene, a CTL signature gene, an antigen presentation/processing signature gene, a tumor inflammation signature gene, or PD-L1 expression. In some embodiments, the biomarker further comprises levels of CD3 and/or Ki67, or CD4, CXCR4, CXCL12, arginase, FAPalpha, CD33 or CD11b. In some embodiments, the biomarker comprises levels of CD8⁺ T cells or CD8⁺ T cells/T_{reg} ratio or granzyme B levels. In some embodiments, such levels are measured by immunohistochemistry staining.

In another aspect, the present invention provides a kit for assaying a tumor sample from a patient treated with a CXCR4 inhibitor optionally in combination with a PD-1 antagonist to obtain normalized RNA expression scores for a gene signature associated with the tumor, wherein the kit comprises:
(a) a set of hybridization probes capable of specifically binding to a transcript expressed by each of the genes; and
(b) a set of reagents designed to quantify the number of specific hybridization complexes formed with each hybridization probe. In some embodiments, the gene signature is selected from two or more of CD8A, CD8B, FoxP3, granzyme B, an IFN-γ signature, a CTL signature, an antigen presentation/processing signature, a tumor inflammation signature, or PD-L1 expression.

In another aspect, the present invention provides a method for treating a patient having a tumor, comprising determining if a sample of the tumor is positive or negative for a biomarker such as a gene signature biomarker and administering to the patient a CXCR4 inhibitor optionally in combination with a PD-1 antagonist if the tumor is positive for the biomarker and administering to the subject a cancer treatment that does not include a CXCR4 inhibitor or PD-1 antagonist if the tumor is negative for the biomarker, wherein the biomarker such as gene signature biomarker is for a biomarker, e.g. gene signature biomarker, that comprises at least two of the clinical response biomarkers selected from CD8⁺ T cells or CD8⁺ T cells/T_{reg} ratio, CD8⁺Ki-67⁺ T cells, granzyme B, an IFN-γ signature score, a CTL signature score, an antigen presentation/processing signature score, a tumor inflammation signature score, a VISTA biomarker panel, or PD-L1 expression. In some embodiments, a multi-gene signature score, such as an IFN-γ, a CTL, an antigen presentation/processing, or a tumor inflammation signature score can be used as one “biomarker” in the same grouping as other individual gene biomarkers, to calculate a more predictive gene signature score.

In another aspect, the present invention provides a method of testing a tumor sample removed from a patient to generate a signature score for a gene signature that is correlated with an anti-tumor response to a CXCR4 inhibitor, optionally in combination with a PD-1 antagonist, wherein the method comprises:
(a) measuring the raw RNA level in the tumor sample for each gene in the gene signature and for each gene in a normalization gene set, wherein the gene signature comprises CD8A, CD8B, FoxP3, granzyme B, an IFN-γ signature score, a CTL signature score, an antigen presentation/processing signature score, a tumor inflammation signature score, a VISTA biomarker panel, or PD-L1;
(b) normalizing the measured raw RNA level for each gene in the gene signature using the measured RNA levels of the normalization genes;
(c) multiplying each normalized RNA value by a calculated scoring weight set to generate a weighted RNA expression value; and
(d) adding the weighted RNA expression values to generate the gene signature score.

In some embodiments, a multi-gene signature score, such as an IFN-γ or CTL signature score, can be used as one “biomarker” in the same grouping as other individual gene biomarkers, to calculate a more predictive gene signature score. In some embodiments, the measuring step comprises isolating RNA from the tissue sample and incubating the tissue sample with a set of probes that are designed to specifically hybridize to gene target regions of the RNA.

Use of CXCR4 Inhibitors and Immunotherapeutic Agents in Treating Cancer

As described in detail below, it has surprisingly been found that treatment of a cancer, such as metastatic melanoma, in a patient with a CXCR4 inhibitor such as X4P-001 or X4-136, optionally in combination with an immunotherapeutic agent such as pembrolizumab, produces a clinical response gene set that correlates with an anti-tumor response in the patient.

Cancer immunotherapy and targeted therapies, such as with ipilimumab or a PD-1 antagonist or antibody, can produce long-lasting responses against metastatic cancer having a wide range of histologies. However, an improved understanding of how some tumors avoid the immune response is required in order to broaden their applicability. It is difficult to study such mechanisms because the interactions between the immune system and cancer cells are continuous and dynamic, meaning that they evolve over time from the initial establishment of the cancer through development of metastasis, which allows the tumor to avoid the immune system. It is now understood that the use of immunotherapy alone may be hindered or rendered ineffective by primary, adaptive, or acquired resistance mechanisms (“immune escape”). See, e.g., Sharma, P. et al., Cell 2017, 168, 707-723 [30].

Recent studies demonstrate that CXCR4/CXCL12 is a primary receptor-ligand pair that cancer cells and surrounding stromal cells use to block normal immune function and promote angiogenesis through the trafficking of T-effector and T-regulatory cells, as well as myeloid derived suppressor cells (MDSCs), in the tumor microenvironment. Cancer cell CXCR4 overexpression contributes to tumor growth, invasion, angiogenesis, metastasis, relapse, and therapeutic resistance. Accordingly, CXCR4 antagonism represents a means to disrupt tumor-stromal interactions, sensitize cancer cells to cytotoxic drugs, and/or reduce tumor growth and metastatic burden.

CXCR4 (C-X-C chemokine receptor type 4) is a chemokine receptor expressed on a wide range of cell types, including normal stem cells, hematopoietic stem cells (HSC), mature lymphocytes, and fibroblasts [1]. CXCL12 (previously referred to as SDF-1α) is the sole ligand for CXCR4. The primary physiologic functions of the CXCL12/CXCR4 axis include the migration of stem cells both during embryonic development (CXCR4−/− knock-out embryos die in utero) and subsequently in response to injury and inflammation. Increasing evidence indicates multiple potential roles for CXCR4/CXCL12 in malignancy. Direct expression of one or both factors has been observed in several tumor types. CXCL12 is expressed by cancer-associated fibroblast (CAFs) and is often present at high levels in the TME. In clinical studies of a wide range of tumor types, including breast, ovarian, renal, lung, and melanoma, expression of CXCR4/CXCL12 has been associated with a poor prognosis and with an increased risk of metastasis to lymph nodes, lung, liver and brain, which are sites of CXCL12 expression [2]. CXCR4 is frequently expressed on melanoma cells, particularly the CD133⁺ population that is considered to represent melanoma stem cells [2, 3] and in vitro experiments and murine models have demonstrated that CXCL12 is chemotactic for those cells [4].

Pembrolizumab is a humanized IgG4 kappa monoclonal antibody that blocks the interaction between PD-1 and its ligands, PD-L1 and PD-L2 [11]. It belongs to the emerging class of immunotherapeutics referred to as checkpoint modulators (CPM). These agents have been developed based on observations that in multiple types of malignancies, the tumor suppresses the host anti-tumor immune response by exploiting counter-regulatory mechanism that normally act as “checkpoints” to prevent the overactivation of the immune system in infection and other situations. In the case of melanoma, PD-L1 is expressed by cells in the TME, engages PD-1, a membrane-associated receptor on CD8⁺ effector T cells, and triggers inhibitory signaling that reduces the killing capacity of cytotoxic T cells.

Pembrolizumab is currently FDA approved for the treatment of unresectable or metastatic melanoma. In a Phase 3 trial, the objective response rate was 33% compared to 12% for ipilimumab (P<0.001) [11]. Analysis of tumor samples before and during treatment in an earlier study demonstrated that a clinical response was associated with an increase in the density of CD8⁺ T cells in the tumor parenchyma (center), while disease progression was associated with persistent low levels of those cells [12]. In an autochthonous murine model of pancreatic adenocarcinoma, persistent tumor growth despite administration of anti-PD-L1 was similarly associated failure of tumor-specific cytotoxic T cells to enter the TME despite their presence in the peripheral circulation [7]. This immunosuppressed phenotype was associated with CXCL12 production by CAF. Moreover, administration of a CXCR4 antagonist (AMD3100) induced rapid T-cell accumulation among the cancer cells and, in combination with anti-PD-L1, synergistically decreased tumor growth.

Multiple observations implicate the CXCL12/CXCR4 axis in contributing to the lack (or loss) of tumor responsiveness to angiogenesis inhibitors (also referred to as “angiogenic escape”). In animal cancer models, interference with CXCR4 function has been demonstrated to disrupt the tumor microenvironment (TME) and unmask the tumor to immune attack by multiple mechanisms, including eliminating tumor re-vascularization [19, 20] and increasing the ratio of CD8⁺ T cells to T_{reg} cells [19, 21,22]. These effects result in significantly decreased tumor burden and increased overall survival in xenograft, syngeneic, as well as transgenic, cancer models [19, 21, 20].

X4P-001, formerly designated AMD11070, is a potent, orally bioavailable CXCR4 antagonist [23], that has demonstrated activity in solid and liquid tumor models [24, and unpublished data] and has previously (under the designations AMD070 and AMD11070) been in Phase 1 and 2a trials involving a total of 71 healthy volunteers [23,25,26] and HIV-infected subjects [27,28]. These studies demonstrated that oral administration of up to 400 mg BID for 3.5 days (healthy volunteers) and 200 mg BID for 8-10 days (healthy volunteers and HIV patients) was well-tolerated with no pattern of adverse events or clinically significant laboratory changes. These studies also demonstrated pharmacodynamic activity, with dose- and concentration-related changes in circulating white blood cells (WBCs); and a high volume of distribution (VL), suggesting high tissue penetrance.

X4-136, formerly designated AMD12118, is also a potent, orally bioavailable CXCR4 antagonist.

Plerixafor (formerly designated AMD3100, now marketed as Mozobil®) is the only CXCR4 antagonist that is currently FDA approved. Plerixafor is administered by subcutaneous injection and is approved for use in combination with granulocyte-colony stimulating factor (G-CSF) to mobilize hematopoietic stem cells (HSCs) to the peripheral blood for collection and subsequent autologous transplantation in patients with non-Hodgkin's lymphoma (NHL) and multiple myeloma (MM).

Both X4P-001 and plerixafor have been studied in murine models of melanoma, renal cell carcinoma, and ovarian cancer and have demonstrated significant anti-tumor activity, including decreased metastasis and increased overall survival [6]. The treatment effect has been associated with decreased presence of myeloid-derived suppressor cells (MDSCs) in the TME and increased presence of tumor-specific CD8⁺ effector cells [7, 8].

In some embodiments, the CXCR4 inhibitor is selected from plerixafor; USL-311 (U.S. Pat. No. 9,353,086), Ulocuplumab (BMS-936564; Kashyap, M. K. et al. Oncotarget 7: 2809-22 (2016)), BL-8040 (BKT-140; Mukhta, E. et al. Mol. Cancer. Ther. 13(2): 275-84 (2014)), T-140 (Jacobson, O. et al. Nuclear Med. 51(11): 1796-1804 (2010), Tamamura, H. et al. FEBS 569: 99-104 (2004)), LY2510924 (Galsky, M. D. et al. Clin. Cancer Res. 20(13): 3581-88 (2014)), TG-0054 (burixafor; NCT00822341), POL6326 (balixafortide; NCT01905475), PRX177561 (Gravina, G. L. et al. Tumor Biol. 39(6):1-17 (2017)), PF-06747143 (Zhang, Y. et al. Sci. Rep. 7: 7305 (2017)), Compound 3 and others (Li, Z. et al. Eur. J. Med. Chem. 149: 30-44 (2017)), GMI-1359 (WO 2016/089872), Compounds Iq, IIj, and others (Bai, R. et al. Eur. J. Med. Chem. 136: 360-71 (2017)), Compound 49b and others (Zhao, H. et al. Bio. Med. Chem Lett. 25(21): 4950-55 (2015)), and F-50067 (515H7; 22nd EORTC-NCI-AACR Symp Molecular Targ Cancer Ther (Berlin), 2010, Abs 225 & 241).

Without wishing to be bound by any particular theory, it is believed that administration of X4P-001 or X4-136 will increase the density of CD8⁺ T cells among the melanoma tumor cells and that this effect will be sustained when X4P-001 or X4-136 is given in combination with an additional cancer therapy such as an immune checkpoint modulator, e.g., pembrolizumab. Because X4P-001 and X4-136 are well-tolerated in the body, and may increase the ability of the body to mount a robust anti-tumor immune response, administering X4P-001 or X4-136 in combination with an additional cancer therapy such as a checkpoint modulator in multiple tumor types may substantially increase the objective response rate, the frequency of durable long-term responses, and overall survival.

It is further believed that such a result would be achieved with comparatively little toxicity since CXCR4-targeted drugs would not be expected to induce cell cycle arrest in bone marrow and other normal proliferating cell populations. Accordingly, the present invention provides significant advantages in treatment outcomes utilizing the low toxicity and effects of the CXCR4 inhibitors X4P-001 and X4-136 on MDSC trafficking, differentiation, and tumor cell gene expression in certain cancers.

CXCR4 antagonism, e.g., by X4P-001 or X4-136, may be used to treat patients with advanced melanoma and other cancers by multiple mechanisms. See WO2017/127811, which is hereby incorporated by reference. In certain embodiments, administration of X4P-001, or X4-136, increases the density of CD8⁺ T cells, thereby resulting in increased anti-tumor immune attack, for example via T cell infiltration of a tumor such as a melanoma tumor. In certain embodiments, administration of X4P-001, or X4-136, additionally decreases neoangiogenesis and tumor vascular supply; and interferes with the autocrine effect of increased expression by tumors of both CXCR4 and its only ligand, CXCL12, thereby potentially reducing cancer cell metastasis.

In some embodiments, patients with advanced forms of cancer, including melanoma, such as metastatic melanoma, or lung cancer, such as metastatic non-small cell lung cancer, are treated with X4P-001 or X4-136, either as a single agent (monotherapy), or in combination with an immune checkpoint inhibitor, such as pembrolizumab. Pembrolizumab is an antibody to PD-1, which binds to the programmed cell death 1 receptor (PD-1), preventing the receptor from binding to the inhibitory ligand PD-L1, and overrides the ability of tumors to suppress the host anti-tumor immune response, dubbed an immune checkpoint inhibitor.

Without wishing to be bound by any particular theory, it is believed that by combining the two medicaments, the patients' treatment outcome can be further improved by increasing the body's ability to mount a robust anti-tumor immune response.

In one aspect, the present invention provides a method of selecting or predicting which melanoma patients from a general population of such patients will be likely (e.g., more likely than average) to benefit from treatment with X4P-001, or X4-136, or pharmaceutically acceptable salts thereof or pharmaceutical composition thereof, optionally in combination with a checkpoint inhibitor such as pembrolizumab. In some embodiments, the method includes co-administering simultaneously or sequentially an effective amount of one or more additional therapeutic agents, such as those described herein. In some embodiments, the method includes co-administering one additional therapeutic agent. In some embodiments, the method includes co-administering two additional therapeutic agents. In some embodiments, the combination of X4P-001, or X4-136, and the additional therapeutic agent or agents acts synergistically to prevent or reduce immune escape and/or angiogenic escape of the cancer. In some embodiments, the patient has previously been administered another anticancer agent, such as an adjuvant therapy or immunotherapy. In some embodiments, the cancer is refractory. In some embodiments, the additional therapeutic agent is pembrolizumab.

The benefit of neoadjuvant chemo- and immunotherapy has been demonstrated in several operable cancers. Compared to adjuvant therapy, neoadjuvant therapy in patients with locally and regionally advanced cancer has several potential benefits, such as (1) reducing the size of the primary and metastatic tumor increases the probability of achieving negative margin resection; (2) tumor exposure to potentially effective systemic therapy is increased while blood and lymphatic vessels remain intact; and (3) collection of pre- and intra-operative samples of tumor tissue following neoadjuvant therapy offers real-time, in vivo assessment of the effects of the therapy on the tumor cells, the tumor microenvironment (TME), and the immune system.

In some embodiments, X4P-001, or X4-136, or pharmaceutically acceptable salts thereof, is administered to a patient in a fasted state.

In some embodiments, the present invention provides a method for treating patients with cancer that presents as a solid tumor, such as melanoma. In some embodiments, the patient has resectable melanoma, meaning that the patient's melanoma is deemed susceptible to being removed by surgery. In other embodiments, the patient has unresectable melanoma, meaning that it has been deemed not susceptible to being removed by surgery.

In some embodiments, the present invention provides a method for treating advanced cancer, such as melanoma or metastatic melanoma, in a patient in need thereof, comprising administering X4P-001, X4-136, or pharmaceutically acceptable salts and/or compositions thereof. In certain embodiments, the patient was previously administered an immune checkpoint inhibitor. In some embodiments, the patient was previously administered an immune checkpoint inhibitor selected from the group consisting of pembrolizumab (Keytruda®, Merck), ipilumumab (Yervoy®, Bristol-Myers Squibb); nivolumab (Opdivo®, Bristol-Myers Squibb) and atezolizumab (Tecentriq®, Genentech). In some embodiments, the cancer became refractory after treatment with the immune checkpoint inhibitor. In some embodiments, the cancer is refractory or resistant to the immune checkpoint inhibitor even though the patient was not previously administered the checkpoint inhibitor.

In certain embodiments, X4P-001 or X4-136 is co-administered with an immune checkpoint inhibitor, such as those described herein. In some embodiments, the immune checkpoint inhibitor is selected from a PD-1 antagonist, a PD-L1 antagonist, and a CTLA-4 antagonist. In some embodiments, X4P-001 or X4-136 is administered in combination with an immunotherapeutic drug selected from ipilimumab (Yervoy®, Bristol-Myers Squibb); atezolizumab (Tecentriq®, Genentech); nivolumab (Opdivo®, Bristol-Myers Squibb); pidilizumab; avelumab (Bavencio®, Pfizer/Merck KgA); durvalumab (Imfinzi®, AstraZeneca); PDR001; REGN2810; or pembrolizumab (Keytruda®, Merck; previously known as MK-3475). In some embodiments, X4P-001 or X4-136 is administered in combination with pembrolizumab.

Other immune checkpoint inhibitors in development may also be suitable for use in combination with X4P-001 or X4-136. These include atezolizumab (Tecentriq®, Genentech/Roche), also known as MPDL3280A, a fully humanized engineered antibody of IgGI isotype against PD-L1, in clinical trials for non-small cell lung cancer, and advanced bladder cancer, such as advanced urothelial carcinoma; and as adjuvant therapy to prevent cancer from returning after surgery; durvalumab (Astra-Zeneca), also known as MEDI4736, in clinical trials for metastatic breast cancer, multiple myeloma, esophageal cancer, myelodysplastic syndrome, small cell lung cancer, head and neck cancer, renal cancer, glioblastoma, lymphoma and solid malignancies; pidilizumab (CureTech), also known as CT-011, an antibody that binds to PD-1, in clinical trials for diffuse large B-cell lymphoma and multiple myeloma; avelumab (Pfizer/Merck KGaA), also known as MSB0010718C, a fully human IgGI anti-PD-L1 antibody, in clinical trials for non-small cell lung cancer, Merkel cell carcinoma, mesothelioma, solid tumors, renal cancer, ovarian cancer, bladder cancer, head and neck cancer and gastric cancer; and PDR001 (Novartis), an inhibitory antibody that binds to PD-1, in clinical trials for non-small cell lung cancer, melanoma, triple negative breast cancer and advanced or metastatic solid tumors.

Other immune checkpoint inhibitors suitable for use in the present invention include REGN2810 (Regeneron), an anti-PD-1 antibody tested in patients with basal cell carcinoma (NCT03132636); NSCLC (NCT03088540); cutaneous squamous cell carcinoma (NCT02760498); lymphoma (NCT02651662); and melanoma (NCT03002376); pidilizumab (CureTech), also known as CT-011, an antibody that binds to PD-1, in clinical trials for diffuse large B-cell lymphoma and multiple myeloma; avelumab (Bavencio®, Pfizer/Merck KGaA), also known as MSB0010718C), a fully human IgGI anti-PD-L1 antibody, in clinical trials for non-small cell lung cancer, Merkel cell carcinoma, mesothelioma, solid tumors, renal cancer, ovarian cancer, bladder cancer, head and neck cancer, and gastric cancer; and PDR001 (Novartis), an inhibitory antibody that binds to PD-1, in clinical trials for non-small cell lung cancer, melanoma, triple negative breast cancer and advanced or metastatic solid tumors. Tremelimumab (CP-675,206; Astrazeneca) is a fully human monoclonal antibody against CTLA-4 that has been in studied in clinical trials for a number of indications, including: mesothelioma, colorectal cancer, kidney cancer, breast cancer, lung cancer and non-small cell lung cancer, pancreatic ductal adenocarcinoma, pancreatic cancer, germ cell cancer, squamous cell cancer of the head and neck, hepatocellular carcinoma, prostate cancer, endometrial cancer, metastatic cancer in the liver, liver cancer, large B-cell lymphoma, ovarian cancer, cervical cancer, metastatic anaplastic thyroid cancer, urothelial cancer, fallopian tube cancer, multiple myeloma, bladder cancer, soft tissue sarcoma, and melanoma. AGEN-1884 (Agenus) is an anti-CTLA4 antibody that is being studied in Phase 1 clinical trials for advanced solid tumors (NCT02694822).

Pembrolizumab (Keytruda®, Merck) is a humanized antibody that targets the programmed cell death (PD-1) receptor. The structure and other properties of pembrolizumab are specified at http://www.drugbank.ca/drugs/DB09037, accessed on Jan. 18, 2016, the disclosure of which is hereby incorporated herein. Pembrolizumab is approved for use in treating unresectable melanoma and metastatic melanoma, and metastatic non-small cell lung cancer in patients whose tumors express PD-1, and have failed treatment with other chemotherapeutic agents. Additionally, pembrolizumab has been tested or mentioned as a possible treatment in other oncologic indications, including solid tumors, thoracic tumors, thymic epithelial tumors, thymic carcinoma, leukemia, ovarian cancer, esophageal cancer, small cell lung cancer, head and neck cancer, salivary gland cancer, colon cancer, rectal cancer, colorectal cancer, urothelial cancer, endometrial cancer, bladder cancer, cervical cancer, hormone-resistant prostate cancer, testicular cancer, triple negative breast cancer, renal cell and kidney cancer, pancreatic adenocarcinoma and pancreatic cancer, gastric adenocarcinoma, gastrointestinal and stomach cancer; brain tumor, malignant glioma, glioblastoma, neuroblastoma, lymphoma, sarcoma, mesothelioma, respiratory papilloma, myelodysplastic syndrome and multiple myeloma.

In a Phase 3 trial in unresectable or metastatic melanoma, the objective response rate was 33% compared to 12% for ipilimumab (P<0.001) [11]. Analysis of tumor samples before and during treatment in an earlier study demonstrated that a clinical response was associated with an increase in the density of CD8⁺ T cells in the tumor parenchyma (center), while disease progression was associated with persistent low levels of those cells [12]. In an autochthonous murine model of pancreatic adenocarcinoma, persistent tumor growth despite administration of anti-PD-L1 was similarly associated failure of tumor-specific cytotoxic T cells to enter the TME despite their presence in the peripheral circulation [7]. This immunosuppressed phenotype was associated with CXCL12 production by CAF. By increasing the density of CD8⁺ T cells among the melanoma tumor cells administration of X4P-001, or X4-136, in combination with pembrolizumab or other checkpoint modulators in multiple tumor types may substantially increase the objective response rate, the frequency of durable long-term responses, and overall survival.

In its current prescribed labeling for unresectable or metastatic melanoma, the recommended course of administration for pembrolizumab is 2 mg/kg as an intravenous infusion over 30 minutes every three weeks. In the discretion of the clinician, depending upon individual tolerance, the prescribed dose of pembrolizumab may be increased to 10 mg/kg every 21 days or 10 mg/kg every 14 days. In the discretion of the clinician, together with the warnings provided with prescribing information, administration of pembrolizumab may be discontinued, or the dose reduced in the case of significant adverse effects.

In some embodiments, the present invention provides a method for treating metastatic melanoma in a patient comprising administering to the patient X4P-001, or X4-136, or pharmaceutically acceptable salts thereof in combination with an immune checkpoint inhibitor. In some embodiments, the melanoma is resectable and metastatic. In other embodiments, the melanoma is unresectable and metastatic. In some embodiments, the immune checkpoint inhibitor is pembrolizumab.

In some embodiments, the present invention provides a method for treating resectable metastatic melanoma in a patient comprising administering to the patient X4P-001, or X4-136, or pharmaceutically acceptable salts thereof in combination with an immune checkpoint inhibitor. After completion of treatment in accordance with the present invention, resection surgery may be performed. In other embodiments, the present invention provides a method for treating unresectable metastatic melanoma in a patient comprising administering to the patient X4P-001, or X4-136, or pharmaceutically acceptable salts thereof in combination with an immune checkpoint inhibitor. In some embodiments, the immune checkpoint inhibitor is pembrolizumab. After completion of treatment in accordance with the present invention, the patient may continue to receive standard of care (SOC) therapy with pembrolizumab or another therapy per the treating clinician's discretion, and such treatment may include further treatment with X4P-001, or X4-136, or pharmaceutically acceptable salts thereof.

In some embodiments, the present invention provides a method for treating a refractory cancer in a patient in need thereof, wherein said method comprises administering to said patient X4P-001, or X4-136, or pharmaceutically acceptable salts thereof in combination with an immune checkpoint inhibitor. In some embodiments, the refractory cancer is metastatic melanoma that expresses PD-L1. In some embodiments, the metastatic melanoma expresses PD-L1 and exhibits disease progression after the patient has undergone chemotherapy or treatment with an immune checkpoint inhibitor but not X4P-001 or X4-136. In some embodiments, the refractory cancer is metastatic non-small cell lung cancer (NSCLC) that expresses PD-L1, and which exhibits disease progression after platinum-containing chemotherapy. In some embodiments, the refractory cancer is metastatic melanoma and the immune checkpoint inhibitor is pembrolizumab.

In some embodiments, a provided method comprises administering X4P-001, or X4-136, or pharmaceutically acceptable salts thereof, to a patient in a fasted state and administering the immune checkpoint inhibitor to a patient in either a fasted or fed state.

In certain embodiments, the present invention provides a method for treating cancer in a patient in need thereof, wherein said method comprises administering to said patient X4P-001, or X4-136, or pharmaceutically acceptable salts thereof in combination with an immune checkpoint inhibitor, further comprising the step of obtaining a biological sample from the patient and measuring the amount of a disease-related biomarker. In some embodiments, the biological sample is a blood sample or skin punch biopsy. In certain embodiments, the disease-related biomarker is circulating CD8⁺ T cells and/or plasma levels of PD-1 and/or PD-L1. In some embodiments, the biomarker one or more of is CD8⁺ T cells or CD8⁺ T cells/T_{reg} ratio, CD8⁺Ki-67⁺ T cells, granzyme B, an IFN-γ signature score, a CTL signature score, an antigen presentation/processing signature score, a tumor inflammation signature score, a VISTA biomarker panel, or PD-L1 expression.

In certain embodiments, the present invention provides a method for treating advanced cancer, such as melanoma or non-small cell lung cancer, in a patient in need thereof, wherein said method comprises administering to said patient X4P-001, or X4-136, or pharmaceutically acceptable salts thereof in combination with pembrolizumab, further comprising the step of obtaining a biological sample from the patient and measuring the amount of a disease-related biomarker. In some embodiments, the biological sample is a blood sample or skin punch biopsy. In certain embodiments, the disease-related biomarker is circulating CD8⁺ T cells and/or plasma levels of PD-1 and/or PD-L1. In some embodiments, the disease-related biomarker is one or more of CD8⁺ T cells or CD8⁺ T cells/T_{reg} ratio, CD8⁺Ki-67⁺ T cells, granzyme B, an IFN-γ signature score, a CTL signature score, an antigen presentation/processing signature score, a tumor inflammation signature score, a VISTA biomarker panel, and/or PD-L1 expression.

In other embodiments of the invention, X4P-001, or X4-136, or pharmaceutically acceptable salts thereof are administered in combination with an immune checkpoint inhibitor. The immune checkpoint inhibitor may be an antibody to PD-1, PD-L1, or CTLA-4. In certain embodiments, the immune checkpoint antagonist is selected from the group consisting of pembrolizumab, nivolumab, and ipilimumab.

In some embodiments, the present invention provides a method of treating cancer in a patient in need thereof, wherein said method comprises administering to said patient X4P-001, or X4-136, or pharmaceutically acceptable salts thereof in combination with an immune checkpoint inhibitor, wherein the X4P-001, or X4-136, or pharmaceutically acceptable salts thereof and the immune checkpoint inhibitor act synergistically. One of ordinary skill in the art will appreciate that active agents (such as X4P-001, or X4-136, and an immune checkpoint inhibitor) act synergistically when the combination of active agents results in an effect that is greater than additive. In some embodiments, the immune checkpoint inhibitor is pembrolizumab.

In some embodiments, the present invention provides a method for sensitizing a cancer in a patient in need thereof, wherein the method comprises administering to said patient a CXCR4 inhibitor, such as X4P-001, or X4-136, or pharmaceutically acceptable salts thereof, in combination with an immune checkpoint inhibitor. In some embodiments, the method comprises administering X4P-001 or X4-136 to the patient prior to treatment with the immune checkpoint inhibitor. In some embodiments, the cancer is a solid tumor. In some embodiments, the method comprises first obtaining from the patient a tumor sample, such as a biopsy of the patient's cancer or solid tumor, a baseline measurement of a biomarker for sensitivity to treatment with an immune checkpoint inhibitor, and comparing the baseline measurement to a pre-established threshold for treatment with an immune checkpoint inhibitor. In a case where the baseline measurement does not meet the pre-established threshold of the biomarker for sensitivity to treatment with an immune checkpoint inhibitor, the patient is treated with a CXCR4 inhibitor such as X4P-001, or X4-136, or pharmaceutically acceptable salts thereof, with the desired effect of altering (e.g., increasing or decreasing, as the case may be) the baseline measurement to achieve an altered measurement that meets the pre-established threshold. After the patient has been treated with X4P-001, or X4-136, or pharmaceutically acceptable salts thereof, and found to meet the pre-established threshold, the patient is subsequently treated with an immune checkpoint inhibitor, such as a PD-1 inhibitor or a PD-L1 inhibitor.

It is also within the present invention for the treating clinician, in his or her discretion, to treat the patient with an immune checkpoint inhibitor, even if the patient's altered measurement does not meet the pre-established threshold, if it is considered that the patient may still benefit from treatment with the immune checkpoint inhibitor. Alternatively, the treating clinician may continue to treat the patient with X4P-001, or X4-136, or pharmaceutically acceptable salts thereof, and continue to monitor the patient's biomarker levels to achieve the pre-established threshold. It is also within the present invention for the treating clinician, in his or her discretion, to alter the treatment plan for the patient, or to discontinue treatment altogether.

Immune checkpoint inhibitors of use in the present invention include, for example, pembrolizumab, nivolumab, atezolizumab, avelumab, durvalumab, ipilumumab, and pidilizumab.

In certain embodiments, the biomarker is PD-L1. In other embodiments, the biomarker comprises a gene signature for a relevant pathway or gene. In certain embodiments, the biomarker comprises a gene signature for interferon gamma (IFN-γ), which may be a gene signature based upon the expression levels some or all of the genes selected from IFN-γ, CXCL9, CXCL10, HLA-DRA, IDO1, or STAT1. In some embodiments, the gene signature comprises all six genes IFN-γ, CXCL9, CXCL10, HLA-DRA, IDO1, and STAT1. In certain embodiments, the pre-established threshold has been incorporated into the prescribing information that is included in the package insert, on the packaging, or on a website associated with the CXCR4 inhibitor or said immune checkpoint inhibitor.

A variety of cancers may be treated as provided by the present invention. In some embodiments, the cancer is selected from hepatocellular carcinoma, ovarian cancer, ovarian epithelial cancer, fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical adenoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In some embodiments, the cancer is selected from hepatocellular carcinoma (HCC), hepatoblastoma, colon cancer, rectal cancer, ovarian cancer, ovarian epithelial cancer, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical adenoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In some embodiments, the present invention provides a method for treating a cancer that presents as a solid tumor, such as a sarcoma, carcinoma, or lymphoma, comprising the step of administering X4P-001, or X4-136, or pharmaceutically acceptable salts thereof, to a patient in need thereof. Solid tumors generally comprise an abnormal mass of tissue that typically does not include cysts or liquid areas. In some embodiments, the cancer is selected from renal cell carcinoma, or kidney cancer; hepatocellular carcinoma (HCC) or hepatoblastoma, or liver cancer; melanoma; breast cancer; colorectal carcinoma, or colorectal cancer; colon cancer; rectal cancer; anal cancer; lung cancer, such as non-small cell lung cancer (NSCLC) or small cell lung cancer (SCLC); ovarian cancer, ovarian epithelial cancer, ovarian carcinoma, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical carcinoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In some embodiments, the cancer is selected from renal cell carcinoma, hepatocellular carcinoma (HCC), hepatoblastoma, colorectal carcinoma, colorectal cancer, colon cancer, rectal cancer, anal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, chondrosarcoma, anaplastic thyroid cancer, adrenocortical carcinoma, pancreatic cancer, pancreatic ductal carcinoma, pancreatic adenocarcinoma, glioma, brain cancer, neurofibromatosis-1 associated malignant peripheral nerve sheath tumors (MPNST), Waldenstrom's macroglobulinemia, or medulloblastoma.

In some embodiments, the cancer is selected from hepatocellular carcinoma (HCC), hepatoblastoma, colon cancer, rectal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical carcinoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In some embodiments, the cancer is hepatocellular carcinoma (HCC). In some embodiments, the cancer is hepatoblastoma. In some embodiments, the cancer is colon cancer. In some embodiments, the cancer is rectal cancer. In some embodiments, the cancer is ovarian cancer, or ovarian carcinoma. In some embodiments, the cancer is ovarian epithelial cancer. In some embodiments, the cancer is fallopian tube cancer. In some embodiments, the cancer is papillary serous cystadenocarcinoma. In some embodiments, the cancer is uterine papillary serous carcinoma (UPSC). In some embodiments, the cancer is hepatocholangiocarcinoma. In some embodiments, the cancer is soft tissue and bone synovial sarcoma. In some embodiments, the cancer is rhabdomyosarcoma. In some embodiments, the cancer is osteosarcoma. In some embodiments, the cancer is anaplastic thyroid cancer. In some embodiments, the cancer is adrenocortical carcinoma. In some embodiments, the cancer is pancreatic cancer, or pancreatic ductal carcinoma. In some embodiments, the cancer is pancreatic adenocarcinoma. In some embodiments, the cancer is glioma. In some embodiments, the cancer is malignant peripheral nerve sheath tumors (MPNST). In some embodiments, the cancer is neurofibromatosis-1 associated MPNST. In some embodiments, the cancer is Waldenstrom's macroglobulinemia. In some embodiments, the cancer is medulloblastoma.

In some embodiments, the present invention provides a method for treating a cancer selected from leukemia or a cancer of the blood, comprising administering to a patient in need thereof an effective amount of X4P-001, or X4-136, or pharmaceutically acceptable salts thereof or pharmaceutical compositions thereof, optionally in combination with an additional therapeutic agent such as those described herein. In some embodiments, the cancer is selected from acute myeloid leukemia (AML), chronic myeloid leukemia (CML), acute lymphocytic leukemia (ALL), chronic lymphocytic leukemia (CLL), or a virally induced leukemia.

In some embodiments, the patient has a resectable solid tumor, meaning that the patient's tumor is deemed susceptible to being removed by surgery. In other embodiments, the patient has an unresectable solid tumor, meaning that the patient's tumor has been deemed not susceptible to being removed by surgery, in whole or in part.

In some embodiments, the cancer is an advanced cancer, such as an advanced kidney cancer or advanced renal cell carcinoma.

Disease-Related Biomarkers

Cancer research is improved by the identification of intratumoral expression patterns for sets of genes, changes in levels of immune-related cells in the tumor microenvironment, or other changes in the tumor microenvironment, referred to herein generally as “biomarkers” or more specifically in relation to gene expression patterns as “gene signatures,” “gene expression biomarkers,” or “molecular signatures,” which are characteristic of particular types or subtypes of cancer, and which are associated with clinical outcomes. If such an association is predictive of a clinical response, the biomarker is advantageously used in methods of selecting or stratifying patients as more (or less, as the case may be) likely to benefit from a treatment regimen disclosed herein. It has now been surprisingly found that levels of CD8⁺ T cells or CD8⁺ T cells/T_{reg} ratio, CD8⁺Ki-67⁺ T cells, granzyme B, an IFN-γ signature score, a CTL signature score, an antigen presentation/processing signature score, a tumor inflammation signature score, a VISTA biomarker panel, and/or PD-L1 expression may be used as biomarkers in a method described herein, such as a method of treating cancer in a patient, diagnosing a cancer in a patient, or predicting patient response to treatment of a cancer such as metastatic melanoma. In some embodiments, the biomarker comprises the RNA expression level of a gene described herein, such as CD8A, CD8B, FoxP3, granzyme B, an IFN-γ signature gene, a CTL signature gene, an antigen presentation/processing signature gene, a tumor inflammation signature gene, or PD-L1 expression. In some embodiments, the biomarker further comprises levels of CD3 and/or Ki67.

It has been surprisingly found that X4P-001 and X4-136 increases granzyme B (GZMB) expression in cancers such as solid tumors, e.g. advanced or metastatic melanoma. Granzyme B is associated with cell death/apoptosis mediated by cytotoxic T lymphocytes (CTLs), natural killer (NK) cells, and cytotoxic T cells. Accordingly, in some embodiments, the biomarker is an observed increase in granzyme B expression in a tumor relative to a control. In some embodiments, the cancer is a solid tumor such as advanced or metastatic melanoma.

It has been surprisingly found that X4P-001 and X4-136 increases observed numbers of CD8⁺ T cells and/or CD4⁺ T cells in cancers such as solid tumors, e.g. advanced or metastatic melanoma. Accordingly, in some embodiments, the biomarker is an observed increase in CD8⁺ T cells and/or CD4⁺ T cells in a tumor relative to a control. In other embodiments, the biomarker is an increase in the ratio of CD8⁺ T cells to Treg cells. In some embodiments, the increase is observed by immunohistochemistry or expression levels of one or both of CD8A and CD8B. In some embodiments, an increase in CD8⁺ T cells and/or CD4⁺ T cells or CD8⁺ T cells/T_{reg} ratio in a tumor sample from a patient who has undergone treatment with X4P-001 or X4-136 correlates with an increased likelihood that the patient will benefit from continued treatment with X4P-001 alone or in combination with an immunotherapeutic agent, e.g., a checkpoint inhibitor such as a PD-1 antagonist. In some embodiments, the PD-1 antagonist is selected from nivolumab and pembrolizumab, or a biosimilar or variant of such PD-1 antagonists. In some embodiments, the checkpoint inhibitor is nivolumab. In some embodiments, the checkpoint inhibitor is a nivolumab biosimilar or variant. In some embodiments, the checkpoint inhibitor is pembrolizumab. In some embodiments, the checkpoint inhibitor is a pembrolizumab biosimilar or variant. In some embodiments, the tumor is a solid tumor such as advanced or metastatic melanoma.

It has been surprisingly found that X4P-001 and X4-136 increase one or more of a panel of IFN-γ related genes referred to herein as an “IFN-γ gene signature.” In some embodiments, the IFN-γ gene signature is selected from a change (i.e. an increase or decrease) of one or more of IDO1, CXCL10, CXCL9, HLA-DRA, STAT1 and IFN-γ, or a net increase or decrease of the group as a whole, in a tumor relative to a control. In some embodiments, the biomarker is IDO1. In some embodiments, the biomarker is CXCL10. In some embodiments, the biomarker is CXCL9. In some embodiments, the biomarker is HLA-DRA. In some embodiments, the biomarker is STAT1. In some embodiments, the biomarker is IFN-γ. In some embodiments, the biomarker is two or more of IDOL, CXCL10, CXCL9, HLA-DRA, STAT1 and IFN-γ. In some embodiments, the biomarker is three or more of IDO1, CXCL10, CXCL9, HLA-DRA, STAT1 and IFN-γ. In some embodiments, the biomarker is four or more of IDOL, CXCL10, CXCL9, HLA-DRA, STAT1 and IFN-γ. In some embodiments, the biomarker is five or more of IDOL, CXCL10, CXCL9, HLA-DRA, STAT1 and IFN-γ. In some embodiments, the biomarker is all of IDOL, CXCL10, CXCL9, HLA-DRA, STAT1 and IFN-γ. In some embodiments, the biomarker is an increase of all of IDO1, CXCL10, CXCL9, HLA-DRA, STAT1 and IFN-γ. In some embodiments, an increase in one, two, three, four, five, or all of IDOL, CXCL10, CXCL9, HLA-DRA, STAT1 and IFN-γ in a tumor sample from a patient who has undergone treatment with X4P-001 correlates with an increased likelihood that the patient will benefit from continued treatment with X4P-001 alone or in combination with an immunotherapeutic agent, e.g., a checkpoint inhibitor such as a PD-1 antagonist. In some embodiments, the PD-1 antagonist is selected from nivolumab and pembrolizumab, or a biosimilar or variant of such PD-1 antagonists. In some embodiments, the checkpoint inhibitor is nivolumab. In some embodiments, the checkpoint inhibitor is a nivolumab biosimilar or variant. In some embodiments, the checkpoint inhibitor is pembrolizumab. In some embodiments, the checkpoint inhibitor is a pembrolizumab biosimilar or variant. In some embodiments, the tumor is a solid tumor such as advanced or metastatic melanoma. In some embodiments, the biomarker or the use thereof is one of those described in Ayers et al., Journal of Clinical Investigation 2017, 127(8), 2930-2940 [29] (“Ayers et al. (2017)”) or WO 2016/094377, each of which is hereby incorporated by reference.

Without wishing to be bound by theory, it is believed that, since a high basal IFN-gamma signature is associated with a higher likelihood of response to a check point inhibitor, if a CXCR4 inhibitor increases the IFN-gamma signature, then CXCR4 treatment increases the likelihood of a tumor's response to checkpoint inhibitor. In some embodiments, treatment with a CXCR4 inhibitor primes the tumor microenvironment such that the tumor becomes more likely to respond to an immunotherapeutic agent. In some embodiments, the tumor does not respond to monotherapy with a PD-1 inhibitor, but becomes primed and responds to the PD-1 inhibitor when combined with a CXCR4 inhibitor. In some embodiments, the tumor initially responds to the PD-1 inhibitor or another checkpoint inhibitor, but becomes refractory. In some embodiments, after treatment with a CXCR4 inhibitor, the tumor can be treated effectively with the PD-1 inhibitor or other immunotherapeutic agent.

In other embodiments the biomarker is two, three, four, five, six, seven, eight, about ten, about twenty, or more of an expanded 28-gene immune signature consisting of: IL2Rg; CXCR6; CD3d; CD2; ITGAL; TAGAP; CIITA; HLA-DRA; PTPRC; CXCL9; CCL5; NKG7; GZMA; PRF1; CCR5; CD3e; GZMK; IFNG; HLA-E; GZMB; PDCD1; SLAMF6; CXCL13; CXCL10; IDO1; LAG3; STAT1; and CXCL11; or an expanded 10-gene IFN-γ signature comprising IFNG, STAT1, CCR5, CXCL9, CXCL10, CXCL11, IDO1, PRF1, GZMA, and MHCII HLA-DRA. Ayers et al. (2017).

In other embodiments the biomarker is one or more of a panel of antigen presentation/procession related genes referred to herein as an “antigen presentation/processing gene signature.” In some embodiments, the antigen presentation/processing gene signature is selected from a change (i.e. an increase or decrease) of one or more of B2M, CD74, CTSL, CTSS, HLA-DMA, HLA-DMB, HLA-DOB, HLA-DPA1, HLA-DPB1, HLA-DQA, HLA-DQB1, HLA-DRA, HLA-DRB1, HLA-DRB3, PSMB8, PSMB9, TAP1, and TAP2, or a net increase or decrease of the group as a whole, in a tumor relative to a control. In some embodiments, an increase in one, two, three, four, five, ten, fifteen, or all of B2M, CD74, CTSL, CTSS, HLA-DMA, HLA-DMB, HLA-DOB, HLA-DPA1, HLA-DPB1, HLA-DQA1, HLA-DQB1, HLA-DRA, HLA-DRB1, HLA-DRB3, PSMB8, PSMB9, TAP1, and TAP2 in a tumor sample from a patient who has undergone treatment with X4P-001 correlates with an increased likelihood that the patient will benefit from continued treatment with X4P-001 alone or in combination with an immunotherapeutic agent, e.g., a checkpoint inhibitor such as a PD-1 antagonist.

In other embodiments the biomarker is one or more of a panel of tumor inflammation related genes referred to herein as a “tumor inflammation gene signature.” In some embodiments, the tumor inflammation gene signature is selected from a change (i.e. an increase or decrease) of one or more of CCL5, CD27, CD274, CD276, CD8A, CMKLR1, CXCL9, CXCR6, HLA-DQA1, HLA-DRB1, HLA-E, IDO1, LAG3, NKG7, PDCD1LG2, PSMB10, STAT1, and TIGIT, or a net increase or decrease of the group as a whole, in a tumor relative to a control. In some embodiments, an increase in one, two, three, four, five, ten, fifteen, or all of CCL5, CD27, CD274, CD276, CD8A, CMKLR1, CXCL9, CXCR6, HLA-DQA1, HLA-DRB1, HLA-E, IDO1, LAG3, NKG7, PDCD1LG2, PSMB10, STAT1, and TIGIT in a tumor sample from a patient who has undergone treatment with X4P-001 correlates with an increased likelihood that the patient will benefit from continued treatment with X4P-001 alone or in combination with an immunotherapeutic agent, e.g., a checkpoint inhibitor such as a PD-1 antagonist.

It has surprisingly been found that X4P-001 and X4-136 treat cancers such as solid tumors, e.g., advanced or metastatic melanoma, without significantly increasing levels of T_{reg} cells. Without wishing to be bound by theory, it is believed that because T_{reg} cells inhibit immune response, this indicates that the tumor microenvironment is exhibiting a significant increase in this immune regulatory response that would normally allow the tumor to evade host immunity. Accordingly, in some embodiments, the biomarker is maintenance or decrease of T_{reg} levels in a tumor relative to a control. In some embodiments, the biomarker is the level of FoxP3 expression, which serves as a means to determine the T_{reg} level. In some embodiments, the biomarker is an increase in the ratio of CD8⁺ T cells/FoxP3 in the tumor microenvironment or tumor sample. In some embodiments, the measured increase of the biomarker in a tumor sample from a patient who has undergone treatment with X4P-001 or X4-136 correlates with an increased likelihood that the patient will benefit from continued treatment with X4P-001, or X4-136, alone or in combination with an immunotherapeutic agent, e.g., a checkpoint inhibitor such as a PD-1 antagonist. In some embodiments, the PD-1 antagonist is selected from nivolumab and pembrolizumab, or a biosimilar or variant of such PD-1 antagonists. In some embodiments, the checkpoint inhibitor is nivolumab. In some embodiments, the checkpoint inhibitor is a nivolumab biosimilar or variant. In some embodiments, the checkpoint inhibitor is pembrolizumab. In some embodiments, the checkpoint inhibitor is a pembrolizumab biosimilar or variant. In some embodiments, the tumor is a solid tumor such as advanced or metastatic melanoma.

It has surprisingly been found that X4P-001 and X4-136 treat cancers such as solid tumors, e.g., advanced or metastatic melanoma, without significantly modulating levels of macrophages in the tumor. Accordingly, in some embodiments, the biomarker is maintenance or approximate maintenance of macrophage levels in the tumor relative to a control.

It has surprisingly been found that X4P-001 and X4-136 increase PD-L1 expression in tumor samples and the tumor microenvironment. Without wishing to be bound by theory, it has been proposed that PD-L1 expressing tumor cells interact with PD-1 expressing T cells to attenuate T cell activation and evasion of immune surveillance, thereby contributing to an impaired immune response against the tumor. Accordingly, in some embodiments, the biomarker is an increase in PD-L1 expression. In some embodiments, increase of the biomarker in a tumor sample from a patient who has undergone treatment with X4P-001 or X4-136 correlates with an increased likelihood that the patient will benefit from continued treatment with X4P-001, or X4-136, alone or in combination with an immunotherapeutic agent, e.g., a checkpoint inhibitor such as a PD-1 antagonist. In some embodiments, the PD-1 antagonist is selected from nivolumab and pembrolizumab, or a biosimilar or variant of such PD-1 antagonists. In some embodiments, the checkpoint inhibitor is nivolumab. In some embodiments, the checkpoint inhibitor is a nivolumab biosimilar or variant. In some embodiments, the checkpoint inhibitor is pembrolizumab. In some embodiments, the checkpoint inhibitor is a pembrolizumab biosimilar or variant. In some embodiments, the tumor is a solid tumor such as advanced or metastatic melanoma.

It has surprisingly been found that X4P-001 and X4-136 increase gene expression of one or more of a panel of cytotoxic T cell (CTL)-related genes referred to herein as a “CTL signature” in tumor samples or the tumor microenvironment. Accordingly, in some embodiments, the biomarker is an increase in the CTL signature. In some embodiments, the CTL signature comprises an increase in one or more of CD8A, CD8B, FLTLG, GZMM, or PRF1. In some embodiments, the CTL signature comprises an increase in two or more, three or more, four or more, or each of CD8A, CD8B, FLTLG, GZMM, or PRF1. In some embodiments, the biomarker is a net increase in total expression of the CTL signature. In some embodiments, increase of the biomarker in a tumor sample from a patient who has undergone treatment with X4P-001 or X4-136 correlates with an increased likelihood that the patient will benefit from continued treatment with X4P-001, or X4-136, alone or in combination with an immunotherapeutic agent, e.g., a checkpoint inhibitor such as a PD-1 antagonist. In some embodiments, the PD-1 antagonist is selected from nivolumab and pembrolizumab, or a biosimilar or variant of such PD-1 antagonists. In some embodiments, the checkpoint inhibitor is nivolumab. In some embodiments, the checkpoint inhibitor is a nivolumab biosimilar or variant. In some embodiments, the checkpoint inhibitor is pembrolizumab. In some embodiments, the checkpoint inhibitor is a pembrolizumab biosimilar or variant. In some embodiments, the tumor is a solid tumor such as advanced or metastatic melanoma.

It has surprisingly been found that X4P-001 and X4-136 modulate levels of the VISTA panel of biomarkers in tumor samples and the tumor microenvironment. As used herein, the “VISTA panel” refers to the combination of CD163, CD206, VISTA, COX-2, CD3, and B7H3 biomarkers. In some embodiments, VISTA is decreased after treatment with a CXCR4 inhibitor, such as X4P-001 or X4P-136, optionally in combination with an immunotherapeutic agent. In some embodiments, VISTA and one or more additional members of the VISTA panel are modulated. In some embodiments, CD3 is increased after treatment with the CXCR4 inhibitor optionally in combination with an immunotherapeutic agent.

In accordance with the present invention, biomarkers may be measured before, during, and/or after treatment with a CXCR4 inhibitor and, optionally, an immunotherapeutic agent, and then correlated with clinical outcomes, response rates, prognoses, or another predictive or interpretative measurement.

The system and methods of the present invention are based in part on a combination of a clinical response biomarker (e.g., gene) set and a normalization biomarker (e.g., gene) set, referred to herein as a “biomarker expression platform,” which is employed as a tool for deriving different sets of genes having pre-treatment intratumoral biomarker, e.g., RNA expression, levels (“biomarker signatures” or “gene signatures”) that are correlated with an anti-tumor response to a CXCR4 inhibitor optionally in combination with a PD-1 antagonist for multiple tumor types. This biomarker expression platform is useful to derive a scoring algorithm that weights the relative contribution of individual biomarkers in a signature to a correlation to generate an arithmetic composite of normalized biomarker levels of all of the biomarkers, such as genes in the gene signature, referred to herein as a “gene signature score.” By comparing gene signature scores and anti-tumor responses obtained for a cohort of patients with the same tumor type of interest and treated with a CXCR4 inhibitor optionally in combination with a PD-1 antagonist, a cut-off score may be selected that divides patients according to having a higher or lower probability of achieving an anti-tumor response to treatment. A predictive signature score for a particular tumor type is referred to herein as a gene signature biomarker. Patients whose tumors test positive for a biomarker signature or gene signature biomarker derived according to the present invention are more likely to benefit from therapy with a CXCR4 inhibitor optionally in combination with a PD-1 antagonist than patients whose tumors test negative for the biomarker signature or gene signature biomarker.

Thus, in a first aspect, the invention provides a method of deriving a gene signature biomarker that is predictive of an anti-tumor response to a CXCR4 inhibitor optionally in combination with a PD-1 antagonist for at least one tumor type of interest. The method comprises: (a) obtaining a pre-treatment tumor sample from each patient in a patient cohort diagnosed with the tumor type; (b) obtaining, for each patient in the cohort, an anti-tumor response value following treatment with the CXCR4 inhibitor optionally in combination with a PD-1 antagonist; (c) measuring the raw RNA levels in each tumor sample for each gene in a gene expression platform, wherein the gene expression platform comprises a set of clinical response genes and a set of normalization genes; (d) normalizing, for each tumor sample, each of the measured raw RNA levels for the clinical response genes using the measured RNA levels of the normalization genes; (e) optionally weighting, for each tumor sample and each gene in a gene signature of interest, the normalized RNA expression levels using a pre-defined multiplication coefficient for that gene; (f) optionally adding, for each tumor sample, the weighted RNA expression levels to generate a gene signature score; and (g) comparing the normalized RNA levels or gene signature scores for all of the tumor samples and anti-tumor response values for all of the patients in the cohort to select a cut-off for the RNA levels or gene signature score, respectively, that divides the patient cohort to meet a target biomarker clinical utility criterion. In an embodiment, the method further comprises designating any tumor sample of the tumor type that has a gene signature score that is equal to or greater than the selected cut-off as biomarker high and designating any tumor sample of the tumor type that has a gene signature score that is below the selected cutoff as biomarker low.

The inventors contemplate that gene signature biomarkers derived using the above method of the invention would be useful in a variety of clinical research and patient treatment settings, such as, for example, to selectively enroll only biomarker high patients into a clinical trial of a CXCR4 inhibitor optionally in combination with a PD-1 antagonist, to stratify the analysis of a clinical trial of a CXCR4 inhibitor optionally in combination with a PD-1 antagonist based on biomarker high or negative status, or to determine eligibility of a patient for treatment with a CXCR4 inhibitor optionally in combination with a PD-1 antagonist.

Thus, in a second aspect, the invention provides a method for testing a tumor sample removed from a patient diagnosed with a particular tumor type for the presence or absence of a gene signature biomarker of anti-tumor response of the tumor type to a CXCR4 inhibitor optionally in combination with a PD-1 antagonist. The method comprises: (a) measuring the raw RNA level in the tumor sample for each gene in a gene expression platform, wherein the gene expression platform comprises a set of clinical response genes and a set of normalization genes; (b) normalizing the measured raw RNA level for each clinical response gene in a pre-defined gene signature for the tumor type using the measured RNA levels of the normalization genes; (c) optionally weighting each normalized RNA value using a pre-defined multiplication co-efficient; (d) optionally adding the weighted RNA expression levels to generate a gene signature score; (e) comparing the normalized RNA level or generated score to a reference score or reference RNA level for the gene signature and tumor type; and (f) classifying the tumor sample as biomarker high or biomarker low; wherein if the generated score is equal to or greater than the reference score or measured RNA level is greater than the reference RNA level, then the tumor sample is classified as biomarker high, and if the generated score is less than the reference score or measured RNA level is less than the reference RNA level, then the tumor sample is classified as biomarker low.

In a third aspect, the invention provides a system for testing a tumor sample removed from a patient diagnosed with a particular tumor type for the presence or absence of a gene signature biomarker of anti-tumor response of the tumor type to a CXCR4 inhibitor optionally in combination with a PD-1 antagonist. The system comprises (i) a sample analyzer for measuring raw RNA expression levels of each gene in a gene expression platform, wherein the gene expression platform consists of a set of clinical response genes and a set of normalization genes, and (ii) a computer program for receiving and analyzing the measured RNA expression levels to (a) normalize the measured raw RNA level for each clinical response gene in a pre-defined gene signature for the tumor type using the measured RNA levels of the normalization genes; (b) optionally weight each normalized RNA value using a pre-defined multiplication co-efficient; (c) optionally add the weighted RNA expression levels to generate a gene signature score; (d) compare the normalized RNA levels or generated score to reference RNA levels or a reference score for the gene signature and tumor type; and (e) classify the tumor sample as biomarker high or biomarker low, wherein if the generated score is equal to or greater than the reference score or normalized RNA levels are greater than the reference levels, then the tumor sample is classified as biomarker high, and if the generated score is less than the reference score or normalized RNA levels are less than the reference levels, then the tumor sample is classified as biomarker low.

In each of the above aspects of the invention, the clinical response genes in the gene expression platform are (a) individually correlated with an anti-tumor response to normalized RNA levels in more than one tumor type and (b) collectively generate a covariance pattern that is substantially similar in each of the tumor types. A first subset of genes in the clinical response gene set exhibit intratumoral RNA levels that are positively correlated with the antitumor response while intratumoral RNA levels for a second subset of genes in the clinical response gene set are negatively correlated with the anti-tumor response. In an embodiment, the clinical response gene set comprises about 2-25 genes.

In some embodiments of any of the above aspects of the invention, the set of normalization genes in the gene expression platform comprises genes which individually exhibit intratumoral RNA levels of low variance across multiple samples of the different tumor types and collectively exhibit a range of intratumoral RNA levels that spans the range of intratumoral expression levels of the clinical response genes in the different tumor types. In some embodiments, the normalization gene set comprises about 10 to 12 genes.

In some embodiments, the biomarker or gene signature or normalization gene set is one of those disclosed in WO 2016/094377, the disclosure of which is hereby incorporated by reference.

Dosage and Formulations

X4P-001 is a CXCR4 antagonist with molecular formula C₂₁H₂₇N₅; molecular weight 349.48 amu; appearance: white to pale yellow solid; solubility: freely soluble in the pH range 3.0 to 8.0 (>100 mg/mL), sparingly soluble at pH 9.0 (10.7 mg/mL) and slightly soluble at pH 10.0 (2.0 mg/mL). X4P-001 is only slightly soluble in water; and has a melting point of 108.9° ΔC.

X4-136 is a CXCR4 antagonist with a molecular formula C₂₁H₃₀N₄; and molecular weight of 338.50 amu.

In certain embodiments, the composition containing X4P-001 or X4-136 is administered orally, in an amount from about 200 mg to about 1200 mg daily. In certain embodiments, the dosage composition may be provided twice a day in divided dosage, approximately 12 hours apart. In other embodiments, the dosage composition may be provided once daily. The terminal half-life of X4P-001 has been generally determined to be between about 12 to about 24 hours, or approximately 14.5 hrs. Dosage for oral administration may be from about 100 mg to about 1200 mg once or twice per day. In certain embodiments, the dosage of X4P-001 useful in the invention is from about 200 mg to about 600 mg daily. In other embodiments, the dosage of X4P-001 useful in the invention may range from about 400 mg to about 800 mg, from about 600 mg to about 1000 mg or from about 800 mg to about 1200 mg daily. In certain embodiments, the invention comprises administration of an amount of X4P-001 of about 10 mg, about 20 mg, about 25 mg, about 50 mg, about 75 mg, about 100 mg, about 125 mg, about 150 mg, about 200 mg, about 250 mg, about 300 mg, about 400 mg, about 450 mg, about 500 mg, about 600 mg, about 650 mg, about 700 mg, about 750 mg, about 800 mg, about 850 mg, about 900 mg, about 950 mg, about 1000 mg, about 1100 mg, about 1200 mg, about 1300 mg, about 1400 mg, about 1500 mg or about 1600 mg.

In some embodiments, a provided method comprises administering to the patient a pharmaceutically acceptable composition comprising X4P-001, or X4-136, wherein the composition is formulated for oral administration. In certain embodiments, the composition is formulated for oral administration in the form of a tablet or a capsule. In some embodiments, the composition comprising X4P-001, or X4-136, is formulated for oral administration in the form of a capsule.

In certain embodiments, a provided method comprises administering to the patient one or more capsules comprising 100-1200 mg X4P-001, or X4-136, active ingredient; and one or more pharmaceutically acceptable excipients.

In certain embodiments, the present invention provides a composition comprising X4P-001, or X4-136, or pharmaceutically acceptable salts thereof, one or more diluents, a disintegrant, a lubricant, a flow aid, and a wetting agent. In some embodiments, the present invention provides a composition comprising 10-1200 mg X4P-001, or X4-136, or pharmaceutically acceptable salts thereof, microcrystalline cellulose, dibasic calcium phosphate dihydrate, croscarmellose sodium, sodium stearyl fumarate, colloidal silicon dioxide, and sodium lauryl sulfate. In some embodiments, the present invention provides a unit dosage form wherein said unit dosage form comprises a composition comprising 10-200 mg X4P-001, or X4-136, or pharmaceutically acceptable salts thereof, microcrystalline cellulose, dibasic calcium phosphate dihydrate, croscarmellose sodium, sodium stearyl fumarate, colloidal silicon dioxide, and sodium lauryl sulfate. In certain embodiments, the present invention provides a unit dosage form comprising a composition comprising X4P-001, or X4-136, or pharmaceutically acceptable salts thereof, present in an amount of about 10 mg, about 20 mg, about 25 mg, about 50 mg, about 75 mg, about 100 mg, about 125 mg, about 150 mg, about 200 mg, about 250 mg, about 300 mg, about 400 mg, about 450 mg, about 500 mg, about 600 mg, about 650 mg, about 700 mg, about 750 mg, about 800 mg, about 850 mg, about 900 mg, about 950 mg, about 1000 mg, about 1100 mg, about 1200 mg, about 1300 mg, about 1400 mg, about 1500 mg or about 1600 mg. In some embodiments, a provided composition (or unit dosage form) is administered to the patient once per day, twice per day, three times per day, or four times per day. In some embodiments, a provided composition (or unit dosage form) is administered to the patient once per day or twice per day.

In some embodiments, the present invention provides a unit dosage form comprising a composition comprising:
(a) X4P-001, or X4-136, or pharmaceutically acceptable salts thereof—about 30-40% by weight of the composition;
(b) microcrystalline cellulose—about 20-25% by weight of the composition;
(c) dibasic calcium phosphate dihydrate—about 30-35% by weight of the composition;
(d) croscarmellose sodium—about 5-10% by weight of the composition;
(e) sodium stearyl fumarate—about 0.5-2% by weight of the composition;
(f) colloidal silicon dioxide—about 0.1-1.0% by weight of the composition; and
(g) sodium lauryl sulfate—about 0.1-1.0% by weight of the composition.

In some embodiments, the present invention provides a unit dosage form comprising a composition comprising:
(a) X4P-001, or X4-136, or pharmaceutically acceptable salts thereof—about 37% by weight of the composition;
(b) microcrystalline cellulose—about 23% by weight of the composition;
(c) dibasic calcium phosphate dihydrate—about 32% by weight of the composition;
(d) croscarmellose sodium—about 6% by weight of the composition;
(e) sodium stearyl fumarate—about 1% by weight of the composition;
(f) colloidal silicon dioxide—about 0.3% by weight of the composition; and
(g) sodium lauryl sulfate—about 0.5% by weight of the composition.

Pembrolizumab has been approved by the FDA for treatment of unresectable or metastatic melanoma or metastatic non-small cell lung cancer, and is generally administered at a dosage of 2 mg/kg as an intravenous infusion over 30 minutes once every 3 weeks. Generally, the amount of pembrolizumab or other immune checkpoint inhibitor useful in the present invention will be dependent upon the size, weight, age and condition of the patient being treated, the severity of the disorder or condition, and the discretion of the prescribing physician.

Inasmuch as it may be desirable to administer a combination of active compounds, for example, for the purpose of treating a particular disease or condition, it is within the scope of the present invention that two or more pharmaceutical compositions, at least one of which contains a compound in accordance with the invention, may conveniently be combined in the form of a kit suitable for co-administration of the compositions. Thus, in some embodiments, the invention provides a kit that includes two or more separate pharmaceutical compositions, at least one of which contains a compound of the invention, and means for separately retaining said compositions, such as a container, divided bottle, or divided foil packet. An example of such a kit is the familiar blister pack used for the packaging of tablets, capsules and the like.

The kit of the invention is particularly suitable for administering different dosage forms, for example, oral and parenteral, for administering the separate compositions at different dosage intervals, or for titrating the separate compositions against one another. To assist compliance, the kit typically includes directions for administration and may be provided with a memory aid.

The examples below explain the invention in more detail. The following preparations and examples are given to enable those skilled in the art to more clearly understand and to practice the present invention. The present invention, however, is not limited in scope by the exemplified embodiments, which are intended as illustrations of single aspects of the invention only, and methods which are functionally equivalent are within the scope of the invention. Indeed, various modifications of the invention in addition to those described herein will become apparent to those skilled in the art from the foregoing description and accompanying drawings. Such modifications are intended to fall within the scope of the appended claims.

The contents of each document cited in the specification are herein incorporated by reference in their entireties.

### EXEMPLIFICATION

### Example 1—Measurement of CD8⁺ T Cells

Assessment of the effectiveness of the present invention can be made in part by measurement of the CD8⁺ T cell population. Expanding or increasing the density of CD8⁺ T cells, such as CD8⁺ T-infiltrating lymphocytes (TIL), can help increase tumor recognition and ultimately tumor cell killing. Dudley et al., (2010) Clin. Cancer Research, 16:6122-6131. CD8⁺ T cells can be detected, isolated and quantified utilizing methods described in Herr et al., (1996), J. Immunol. Methods 191:131-142; Herr et al., (1997) J. Immunol. Methods 203:141-152; and Scheibenbogen et al., (2000) J Immunol. Methods 244:81-89. The full disclosure of each of these publications is hereby incorporated by reference herein.

### Example 2—Criteria for Evaluating Response in Patients with Solid Tumors

The response of patients with solid tumors to treatment can be evaluated using the criteria set forth in RECIST 1.1, Eisenhauer et al., (2009) Eur. J. Cancer, 45:228-247, the full disclosure of which is hereby incorporated by reference herein.

### Example 3—Human Melanoma Xenozraft Model

In order to assess the effects of the present invention on the presence of human CD8⁺ effector T cells, accumulation of T_{reg}s in the tumor microenvironment and, ultimately, the effects on metastatic melanoma, a human melanoma xenograft model can be used, as described in Spranger et al. (2013) Sci. Transl. Med., 5:200ra116. A human immune engrafted model may also be used.

### Example 4—Clinical Treatment Regimen—Resectable or Unresectable Metastatic Melanoma

Treatment with X4P-001 as a monotherapy, or in combination with a checkpoint inhibitor, such as pembrolizumab, may be performed in cycles, such as on a 3 week or 9 week cycle. In certain embodiments, the cycle is 9 weeks long. X4P-001 at a determined dose from 200 mg to 1200 mg daily is administered orally either once daily or twice daily in divided doses. Patients are instructed about both dosing schedule and requirements relating to food or drink near the time of dosing.

Dosing Schedule. The daily dose is taken first thing in the morning. Where the dose is divided, the first daily dose is taken in the morning and the second daily dose approximately 12 hours later using the following guidelines:
Dosing should be at the same time(s) each day±2 hr.
For twice daily dosing, the interval between successive doses should not be <9 hours nor >15 hours. If the interval would be >15 hrs, the dose should be omitted and the usual schedule resumed at the next dose.
Restrictions relating to food. Absorption is impacted by food and patients will be instructed as follows:
For the morning dose
   No food or drink (except water) after midnight until the time of dosing
   No food or drink (except water) for 2 hour after dosing.
For the second daily dose, if applicable
   No food or drink (except water) for 1 hour before dosing
   No food or drink (except water) for 2 hours after dosing.

Pembrolizumab is administered consistent with prescribed labeling information. Concomitant treatment with X4P-001 and pembrolizumab may be administered, beginning with daily administration of X4P-001 at day 1. Initial treatment with pembrolizumab is at 2 mg/kg administered by intravenous infusion over 30 minutes in clinic at the week 4 and 7 visits. Patients may, with the approval of their clinician, vary the dosing schedule or dosage of pembrolizumab.

Dosing of X4P-001 and/or pembrolizumab may be adjusted by the clinician as appropriate. The dose of X4P-001 and/or pembrolizumab may be lowered according to the judgment of the clinician. If a patient receiving X4P-001 in combination with pembrolizumab experiences an adverse event at Grade >2, the dose of X4P-001 and/or pembrolizumab may be lowered according to the judgment of the clinician. If a patient successfully completes the first 4 weeks of treatment, that is, without experiencing any adverse events greater than Grade 2, the daily dose of X4P-001 and/or pembrolizumab may be increased, consistent with the judgment of the clinician.

Patients with resectable metastatic melanoma, after combination treatment with X4P-001 and pembrolizumab, will typically undergo complete resection, or resection that is as complete as possible, and could continue to be monitored for recurrence, and/or undergo standard of care (SOC) treatment. This could mean continued use of pembrolizumab, or it could mean some other treatment at the clinician's discretion. Patients with unresectable metastatic melanoma, after treatment, will continue to undergo SOC treatment. Such SOC treatment may or may not include a further regimen of X4P-001, with or without pembrolizumab.

### Evaluation of Response to Treatment and Disease Status

Baseline radiologic assessment of the patient is conducted in order to confirm whether the patient has resectable disease. At end of treatment, repeat imaging will be performed using the same modality.

At initial assessment, the patient is diagnosed as having malignant melanoma, including Stage III (any substage) or Stage IV (with isolated skin metastasis only). Patient is assessed for cutaneous/subcutaneous lesions, including those that will be biopsied clinically.

Cutaneous/subcutaneous lesions ≥3 mm are assessed clinically by the investigator, including the number, distribution, and a description of the lesions (e.g. nodular, popular, macular, pigmented, etc.). The size of the cutaneous lesions is determined using photographs of the lesions (including a ruler with patient study identification and date) obtained as indicated in the schedule of events. Lymph nodes are examined at each visit and the location and size of palpable nodes recorded.

Clinical assessments of cutaneous/subcutaneous disease are conducted at each of day 1, week 4 and week 7, and as indicated based on new signs, symptoms or laboratory findings. Assessments will include physical examination (including lymph nodes) and photographs of all cutaneous lesions, including a ruler marked with patient study number and date.

### Biomarker Assessments

If desired, pharmacokinetic assessment of blood samples for plasma levels of X4P-001 and pembrolizumab may be conducted. Blood samples are collected as scheduled. For example, samples may be taken at day 1, week 4 and week 7. Samples are analyzed for X4P-001 concentration using reversed-phase high performance liquid chromatography (RP-HPLC) with MS/MS detection. The validated range of this bioanalytic method is 30 to 3,000 ng/mL in plasma.

The initial measurement at day 1 is designated as baseline. At week 4 and week 7, measurements of CD8⁺ T cells are taken and compared to baseline.

A primary comparison is the density of specific cell phenotypes in the tumor microenvironment in the pre-treatment biopsy vs. the Week 4 and EOT biopsies. CD8⁺ T cells/mm⁻² are measured in melanoma tumor parenchyma prior to treatment.

An increase at week 4 compared to baseline is considered to be a positive response.

Secondary analyses include (a) comparison of cell phenotypes in the Week 4 vs. EOT biopsies, (b) changes over time in phenotypes among peripheral blood mononuclear cells (PBMCs and in serum biomarker levels. Normally distributed continuous variables are analyzed using t-test and ANOVA/ANCOVA, as appropriate. Variables whose results are not normally distributed are analyzed by non-parametric statistics. Fisher's exact test is used for categorical variables.

Pharmacokinetic assessment of pembrolizumab may be accomplished using techniques, such as those described in Patnaik et al. (2015) Clin. Cancer Res. 21:4286-4293, the full disclosure of which is hereby specifically incorporated herein by reference.

### Example 5—Measurement of Biomarkers

Single Marker and Multiplex Immunofluorescence (mIF)

Single-marker IHC (CD8 and granzyme B) and multiplex IHC staining were analyzed using HALO™ spatial analysis tools, and the entire tumor area of each specimen was scored. (See Tunstall, “Quantifying Immune Cell Distribution in the Tumor Microenvironment Using HALO™ Spatial Analysis Tools,” Application Note, July 216, (Indica Labs), accessed Nov. 1, 2017, on https://thepathologist.com/fileadmin/issues/App_Notes/0016-010-halo-app-note.pdf). See also Sherry et al., “Utilizing multiplex chromogenic IHC and digital image analysis to evaluate immune cell content and spatial distribution within NSCLC tumor tissue” Cancer Research (2017) 77(13) Supp: Abstract 2937. CD8 was measured using a mouse monoclonal antibody (DAKO catalog #M7103, lot #20029542). A Leica Bond RX Autostainer was used following standard protocols. For granzyme B, a mouse monoclonal antibody was used (DAKO #M7235). A Leica Bond RX Autostainer was used following standard protocols.

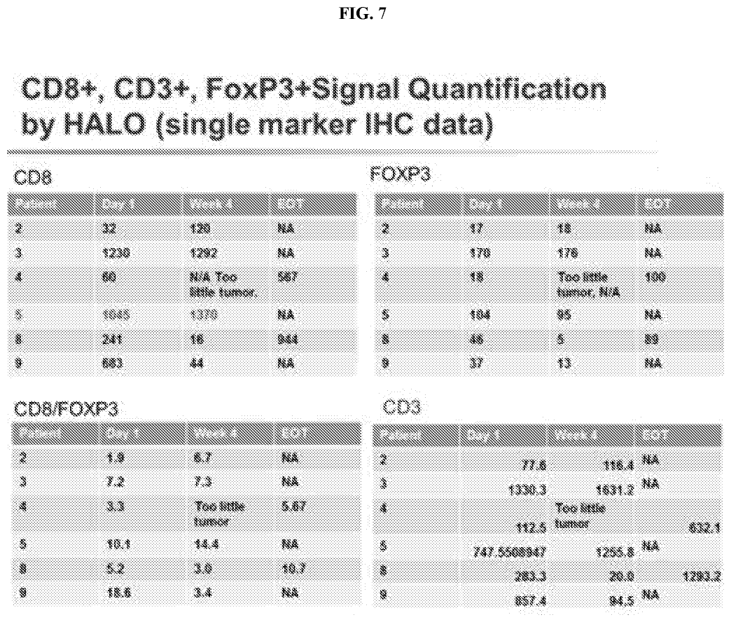

Single-marker IHC was also used to measure CD3, FoxP3, and Ki67. For CD3, a rabbit polyclonal antibody was used (DAKO catalog #A0452, lot #20020069). A Leica Bond RX Autostainer was used following standard protocols. For FoxP3, a mouse monoclonal antibody was used (Abcam catalog #ab20034, lot #GR251424-1). A Leica Bond RX Autostainer was used following standard protocols. For Ki67, a rabbit monoclonal antibody was used (Abcam catalog #ab16667, lot #GR266207-2). A Leica Bond RX Autostainer was used following standard protocols. FIG. 7 shows signal quantification of single marker immunohistochemistry (IHC) data for biomarkers CD8⁺, CD3⁺, and FoxP3 obtained by HALO.

### Patient Evaluation

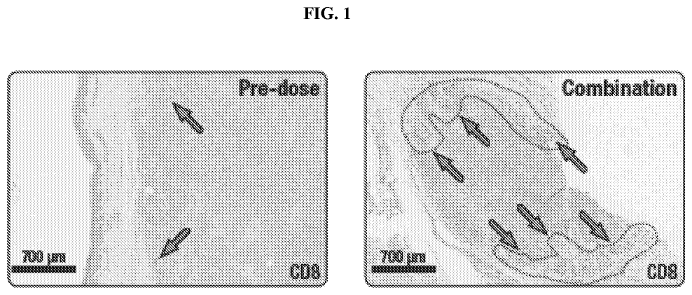

As shown in FIG. 1, photographs of a metastatic melanoma human tumor sample stained with CD8⁺ single-marker IHC stain showed a large increase in CD8⁺ T cell infiltration into the tumor microenvironment after dosing with a combination of X4P-001 and pembrolizumab.

Multiplex Immunofluorescence (mIF)

Formalin-fixed paraffin-embedded (FFPE) tissue sections were baked for 1 hour at 60° C. The slides were dewaxed and stained on a Leica BOND Rx stainer (Leica, Buffalo Grove, Ill.) using Leica Bond reagents for dewaxing (Dewax Solution), antigen retrieval and antibody stripping (Epitope Retrieval Solution 2), and rinsing after each step (Bond Wash Solution). A high stringency wash was performed after the secondary and tertiary applications using high-salt TBST solution (0.05 M Tris, 0.3M NaCl, and 0.1% Tween-20, pH 7.2-7.6). OPAL Polymer HRP Mouse plus Rabbit (PerkinElmer, Hopkington, Mass.) was used for all secondary applications.

TABLE 1
AIR-5 Panel

| | | Clone/ | Company/ | | OPAL |
|---|---|---|---|---|---|
| Position | Antibody | Host | Item | Concentration | Fluor |
| 1 | CD4 | SP35/ | Cell Marque/ | 0.15 ug/mL | 520 |
| | | Rabbit | 104R-16 | | |
| 2 | CD8 | 144B/ | DAKO/ | 0.05 ug/mL | 540 |
| | | Mouse | M7103 | | |
| 3 | PD-1 | D4W2J/ | Cell | 0.06 ug/mL | 570 |
| | | Rabbit | Signaling/ | | |
| | | | 86163 | | |
| 4 | PD-L1 | E1L3N/ | Cell | 2.2 ug/mL | 620 |
| | | Rabbit | Signaling/ | | |
| | | | 13684 | | |
| 5 | CD163 & | EP324/ | BioSB/BSB | 0.125 ug/mL | 650 |
| | CD68 | Rabbit | 3276 | 0.04 ug/mL | |
| | | PG-M1/ | DAKO/ | | |
| | | Mouse | M0876 | | |
| 6 | FoxP3 | 236A/E7/ | eBioscience/ | 5 ug/mL | 690 |
| | | Mouse | 14-4777-82 | | |

Antigen retrieval and antibody stripping steps were performed at 100° C. with all other steps at ambient temperature. Endogenous peroxidase was blocked with 3% H₂O₂ for 8 minutes followed by protein blocking with TCT buffer (0.05 M Tris, 0.15 M NaCl, 0.25% Casein, 0.1% Tween 20, pH 7.6+/−0.1) for 30 minutes. The first primary antibody (position 1) was applied for 60 minutes followed by the secondary antibody application for 10 minutes and the application of the tertiary TSA-amplification reagent (PerkinElmer OPAL fluor) for 10 minutes. The primary and secondary antibodies were stripped with retrieval solution for 20 minutes before repeating the process with the second primary antibody (position 2) starting with a new application of 3% H₂O₂. The process was repeated until all 6 positions were completed; however, there was no stripping step after the 6^{th} position. Slides were removed from the stainer and stained with Spectral DAPI (Perkin Elmer) for 5 minutes, rinsed for 5 minutes, and coverslipped with Prolong Gold Antifade reagent (Invitrogen/Life Technologies, Grand Island, N.Y.).

Slides were cured for 24 hours at room temperature, then representative images from each slide were acquired on PerkinElmer Vectra 3.0 Automated Imaging System. Images were spectrally unmixed using PerkinElmer inForm software and exported as multi-image TIFF's for analysis in HALO software (Indica Labs, Corrales, N. Mex.).

After all fluorescence images were acquired, the coverslips were gently removed by soaking the slides in Bond Wash Solution overnight before placing the wet slides onto the Leica BOND Rx stainer for chromogenic staining using the Leica Bond Polymer Refine Detection kit (Leica #DS9800); however, a TCT 10-minute blocking step was added before the 60-minute primary antibody incubation. Slides were cover-slipped with Cytoseal XYL (Richard-Allan Scientific, Kalamazoo, Mich.), and 20× images were acquired on the Aperio AT Turbo scanning microscope (Leica Biosystems, NuBloch, Germany).

TABLE 2
Chromogenic Stains

| | Clone/ | Company/ | | |
|---|---|---|---|---|
| Antibody | Host | Item | Concentration | Chromogen |
| Melanoma | M2-7C10; | Novus/ | 0.2 μg/mL | DAB |
| Cocktail | M2-9E3, | NBP2- | | |
| | T311, | 34337 | | |
| | HMB45/ | | | |
| | Mouse | | | |

Cellular analysis of the images were then analyzed with HALO image analysis software (Indica Labs, Cooales, N. Mex.). After the cells were visualized based on nuclear recognition (DAPI stain), the software measured fluorescence intensity of the estimated cytoplasmic areas of each cell. A mean intensity threshold above background was used to determine positivity for each fluorochrome within the cytoplasm, thereby, defining cells as either positive or negative for each marker. The positive cell data was then used to define colocalized populations and to perform nearest neighbor spatial analysis.

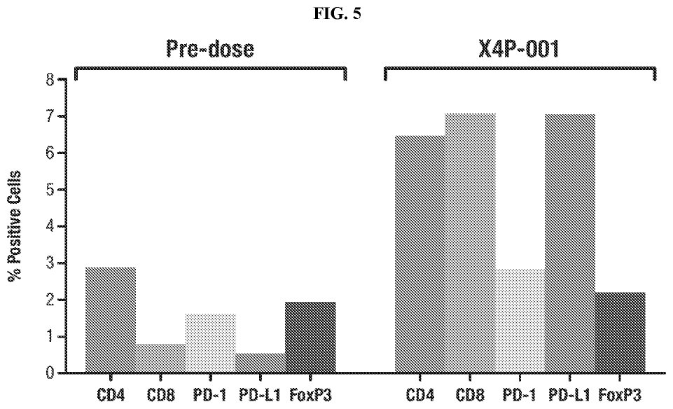

FIG. 5 shows a bar graph of mIF results for melanoma patient #5 demonstrating that treatment with X4P-001 increased the percentage of CD4, CD8, PD-1, and PDL-1 positive cells in the TME. The percentages of Treg (FoxP3⁺) cells and macrophages (CD68/CD163⁺; 24.1% vs. 25.4%; not shown) were not altered.

Formalin-fixed paraffin-embedded melanoma samples were stained sequentially with a 6-component immunophenotyping antibody panel, including CD4, CD8, PD-1, PD-L1, macrophage cocktail (CD68+CD163), and FoxP3 (Tregs). DAPI was used as a nuclear counterstain. Antibodies were detected using HRP-catalyzed deposition of fluorescent tyramide substrates (Opal, Perkin-Elmer). Images were obtained using spectral imaging, autofluorescence subtraction and unmixing (Vectra 3.0, Perkin-Elmer), and analyzed using HALO™ image analysis software.

### Granzyme B and CD8+ T Cell Assessments:

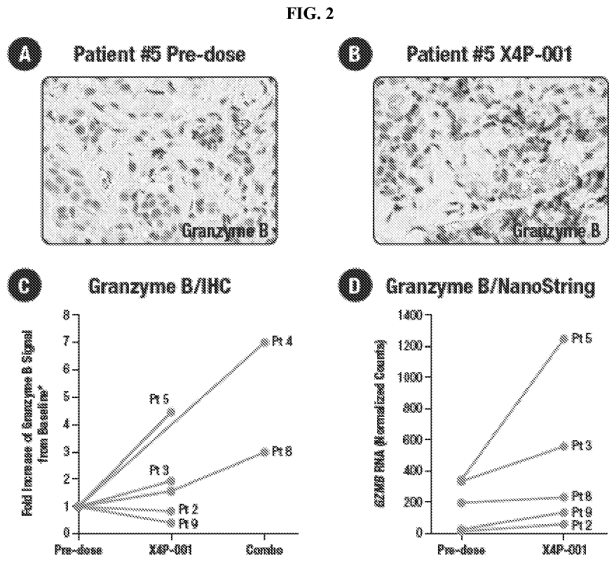

Representative granzyme B IHC staining is shown at baseline (FIG. 2, panel A) and following 21 days of X4P-001 treatment (FIG. 2, panel B). FIG. 2, panel C shows the fold change of granzyme B positivity post-treatment for all evaluable samples. Quantification was performed using HALO™ software and the entire tumor area was scored. FIG. 2, panel D shows the granzyme B RNA expression level for 5 patients with both pre- and post-X4P-001 single agent treatment evaluable biopsies. The RNA expression data in panel D was obtained using NanoString as described herein.

### CD8+ IHC Staining for Single Patient

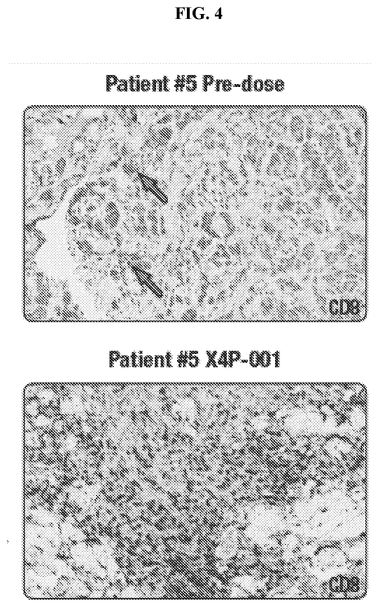

FIG. 4 shows the results of mIF CD8 staining for patient #5 pre- and post-dosing with X4P-001. CD8 expression was visibly increased after dosing.

### Biomarker Investigation Using Nanostring

### Materials and Methods

FFPE gene expression analysis: For each gene biomarker, RNA was extracted from FFPE slides using Qiagen's AllPrep kit (Cat. 80234) and analyzed using NanoString nCounter platform with the PanCancer Immune probe set. Raw counts were normalized using the geometric mean of 30 housekeeping genes and the normalized data from both panels were merged and analyzed with nSolver software (Version 4.0).

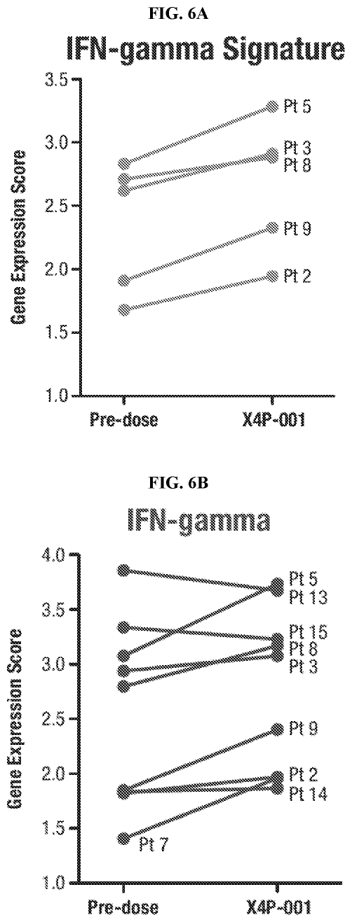

Interferon gene signature was based on Ayers et al (JCI 2017) and calculated in the following manner. For each patient (pt) sample, the geometric mean was calculated from the normalized counts for six genes (IFN-γ, CXCL9, CXCL10, HLA-DRA, IDO1, STAT1) and then the mean was Log 10-transformed to generate the Gene Expression score. FIG. 6 shows gene expression scores pre- and post-dosing with X4P-001 for the interferon gamma (IFN-γ) gene signature. Gene scores were calculated for each patient sample from the geometric mean of normalized counts for IFN-gamma, CXCL9, CXCL10, HLA-DRA, IDO1, and STAT1. The mean was Log 10-transformed to generate the Gene Expression score. The Gene Expression Score increased for each one of the five patients.

The tumor inflammatory signature (TIS) was calculated from 18 genes by taking the Log 10 of the geometric mean of the normalized counts across each gene set to generate a “Gene signature score”. See, e.g., Righi E, Kashiwagi S, Yuan J, et al. “CXCL12/CXCR4 Blockade Induces Multimodal Antitumor Effects That Prolong Survival in an Immunocompetent Mouse Model of Ovarian Cancer,” Cancer Res. 2011; 71(16):5522-5534.

### Results

X4P-001 Increased the IFN-Gamma Gene Expression Signature: NanoString nCounter analysis was conducted with the PanCancer Immune probe set using RNA extracted from FFPE slides. Raw counts were normalized using the geometric mean of housekeeping genes. The Interferon-gamma gene signature score was assessed by a procedure essentially as described in Ayers et al. (2017) J. Clin. Invest. 127:2930-2940.

X4P-001 Increased the CTL Gene Expression Signature: The CTL gene expression signature includes the expression of CD8A, CD8B, FLTLG, GZVIA, and PRF1. To perform the CTL signature measurement, RNA was extracted from FFPE slides using Qiagen's AllPrep kit and analyzed using the NanoString nCounter platform with the PanCancer Immune probe set. Raw counts were normalized using the geometric mean of housekeeping genes. NanoString nCounter validation is described at Malkov et al. (2009) BMC Research Notes; 2:80; accessed Nov. 2, 2017 at https://bmcresnotes.biomedcentral.com/articles/10.1186/1756-0500-2-80; Waggott et al. (2012) Bioinformatics 28:1546-1548; see also nCounter® Analysis System User Manual (July 2015), published by NanoString Technologies®, Inc., accessed Nov. 2, 2017, at https://www.nanostring.com/application/files/7114/8942/6665/MAN-C0035-05_nCounter Analysis_System_GEN2.pdf.

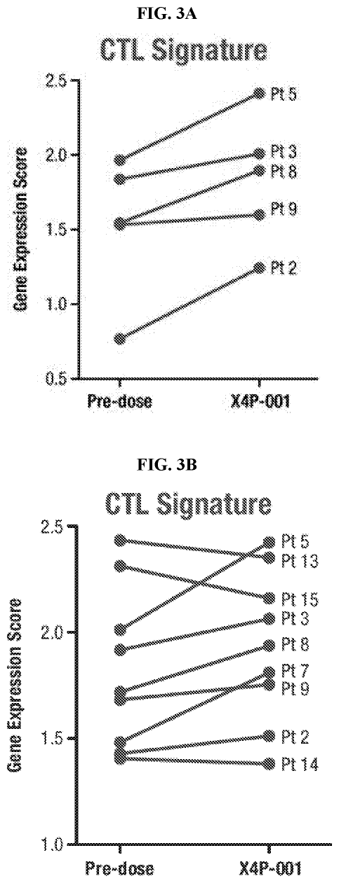

FIG. 3 shows gene expression scores pre- and post-dosing with X4P-001 for the cytotoxic T lymphocyte (CTL) gene signature. Gene scores were calculated for each patient sample from the geometric mean of normalized counts for CD8A, CD8B, FLTLG, GZAM, and PRF1. The mean was Log 10-transformed to generate the Gene Expression score. The gene expression score increased for each one of the five patients.

X4P-001 Effect on CD8A, CD8B, Granzyme B Gene, and FoxP3: Using extraction and NanoString nCounter methods similar to those described above, increased CD8A and CD8B expression were observed; increased granzyme B expression; and similar or unchanged levels of FoxP3.

### Example 6—Nine Week Monotherapy and Combination Therapy Study in Patients with Malignant Melanoma with Measurement of Biomarkers

### Clinical Protocol

A total of sixteen (16) patients were enrolled in a controlled study. The study population was comprised of male and female adult subjects (≥18 years of age) with histologically confirmed malignant melanoma. Subjects were further required to have at least two (2) separate cutaneous or subcutaneous lesions suitable for punch biopsies (≥3 mm).

Subjects were excluded if they had an Eastern Cooperative Oncology Group (ECOG) performance score of two (2) or greater. Subjects were further excluded is they had previously received checkpoint inhibitor therapies (e.g., anti-CTLA-4, PD-1, PD-L1) or oncolytic virus therapy. Subjects with ongoing HIV, hepatitis C, or uncontrollable infections were excluded, as were subjects who had myocardial infarctions, grade three (3) or higher hemorrhage, chronic liver disease, or other active malignancies within the previous six (6) months.

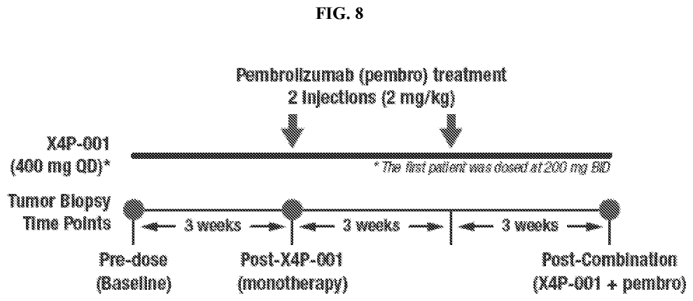

Subjects were first screened and evaluated for baseline measurements. Enrolled participants received treatment a cycle involving a first period comprising X4P-001 monotherapy and a second period comprising of X4P-001 and a checkpoint inhibitor combination therapy. The dosing schedule for the study is summarized in FIG. 8.

Prior to treatment two (2) baseline serum samples were collected from each patient. One baseline serum sample was collected at the time of screening and another was collected one to four weeks later on Day 1 of the treatment, prior to the administration of the first dose of X4P-001. In addition to the baseline serum samples, a baseline punch biopsy was collected from each patient on D1 prior to the administration of X4P-001.

Beginning on Day 1 subjects received 400 mg of X4P-001 orally, q.i.d. One patient received 200 mg orally, b.i.d. Patients were administered X4P-001 throughout the nine (9) week study.

Three (3) weeks after treatment was initiated, additional serum samples were collected from each patient. Additional biopsy samples were also collected unless the attending physician recommended against the biopsy. Following sample collection, subjects were administered the first of two doses of pembrolizumab (2 mg/kg, i.v.).

Three (3) weeks after the administration of the first dose of pembrolizumab (six weeks from beginning of treatment) additional serum samples were collected from each patient. Subjects then administered a second dose of pembrolizumab (2 mg/kg, i.v.).

Three (3) weeks after the administration of the second dose of pembrolizumab (nine weeks from the beginning of treatment) additional serum samples were collected. Additional biopsy samples were also collected unless the attending physician recommended against the biopsy.

### Multiplex Immunofluorescence

Tumor samples obtained from melanoma patients were formalin-fixed and paraffin-embedded (FFPE) according to known procedures. FFPE tissue sections were baked for 1 hour at 60° C. The slides were dewaxed and stained on a Leica BOND Rx stainer (Leica, Buffalo Grove, Ill.) using Leica Bond reagents for dewaxing (Dewax Solution), antigen retrieval and antibody stripping (Epitope Retrieval Solution 2), and rinsing after each step (Bond Wash Solution). A high stringency wash was performed after the secondary and tertiary applications using high-salt TBST solution (0.05 M Tris, 0.3M NaCl, and 0.1% Tween-20, pH 7.2-7.6).

Multiplex IHC staining was analyzed using HALO™ spatial analysis tools, and the entire tumor area of each specimen was scored. (See Tunstall, “Quantifying Immune Cell Distribution in the Tumor Microenvironment Using HALO™ Spatial Analysis Tools,” Application Note, July 216, (Indica Labs), accessed Nov. 1, 2017, on https://thepathologist.com/fileadmin/issues/App_Notes/0016-010-halo-app-note.pdf). See also Sherry et al., Cancer Research 77(13) Supp: Abstract 2937 (2017).

Slides were sequentially stained with antibody panels after rounds of heat-induced epitope retrieval and detected by antibody-binding HRP-containing polymers in conjunction with fluorescent tyramide substrate (Opal, Perkin-Elmer). DAPI was used as a nuclear counterstain. Fluorochromes with spectral overlap were imaged using spectral deconvolution and autofluorescence-subtraction (Vectra 3.0, Perkin-Elmer). Whole-slide scans were imaged using the Aperio-FL System (Leica Biosystems) and transferred into Halo for quantitative digital image analysis. After the cells were visualized based on nuclear recognition (DAPI stain), the software measured fluorescence intensity of the estimated cytoplasmic areas of each cell. A mean intensity threshold above background was used to determine positivity for each fluorochrome within the cytoplasm, thereby, defining cells as either positive or negative for each marker. The positive cell data was then used to define colocalized populations and to perform nearest neighbor spatial analysis.

Staining for CD4, CD8, PD-1, PD-L1, CD163/CD68 (macrophage), FoxP3, Ki-67, and melanoma cells was accomplished using the antibodies listed in TABLE 3 and 4. OPAL Polymer HRP Mouse plus Rabbit (Perkin-Elmer, Hopkington, Mass.) was used for all secondary applications.

TABLE 3
Antibodies for Cell Marker Visualization

| | Clone/ | Company/ | | OPAL |
|---|---|---|---|---|
| Marker | Host | Item | Concentration | Fluor |
| CD4 | SP35/ | Cell Marque/ | 0.15 μg/mL | 520 |
| | Rabbit | 104R-16 | | |
| CD8 | 144B/ | DAKO/M7103 | 0.157 μg/mL | 520 |
| | Mouse | | | |
| PD-1 | D4W2J/ | Cell Signaling/ | 0.06 μg/mL | 570 |
| | Rabbit | 86163 | | |
| PD-L1 | E1L3N/ | Cell Signaling/ | 2.2 μg/mL | 620 |
| | Rabbit | 13684 | | |
| CD163 & | EP324/ | BioSB/BSB 3276 | 0.125 μg/mL | 650 |
| CD68 | Rabbit | DAKO/M0876 | 0.04 μg/mL | |
| | PG-M1/ | | | |
| | Mouse | | | |
| FoxP3 | 236A/E7/ | eBioscience/14- | 5 μg/mL | 690 |
| | Mouse | 4777-82 | | |
| Ki-67 | MIB-1/ | DAKO/M7240 | 0.23 μg/mL | 570 |
| | Mouse | | | |
| Melanoma | M2-7C10; | Novus/NBP2-34337 | 0.03 μg/mL | 650 |
| Cocktail | M2-9E3, | | | |
| | T311, | | | |
| | HMB45/ | | | |
| | Mouse | | | |

TABLE 4
IF

| | Clone/ | Company/ | | OPAL |
|---|---|---|---|---|
| Marker | Host | Item | Concentration | Fluor |
| CD8 | 144B/ | DAKO/M7103 | 0.157 μg/mL | 520 |
| | Mouse | | | |
| Ki67 | MIB-1/ | DAKO/M7240 | 0.23 μg/mL | 570 |
| | Mouse | | | |
| Melanoma | M2-7C10; | Novus/NBP2-34337 | 0.03 μg/mL | 650 |
| Cocktail | M2-9E3, | | | |
| | T311, | | | |
| | HMB45/ | | | |
| | Mouse | | | |

### FoxP3 and CD8 T Cell Ratio Post X4P-001 Single Agent Treatment

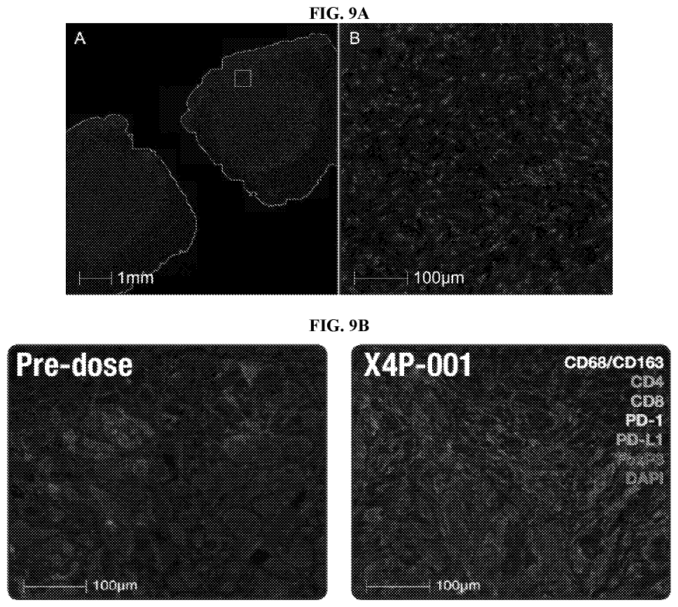

Biopsy samples collected after X4P-001 monotherapy were stained with six (6) antibodies after rounds of heat-induced epitope retrieval to detect CD8, FoxP3, PD-L1, PD-1, melanoma cells, and CD4. Representative CD8 and FoxP3 staining is shown in FIG. 9, Panels A and B. Panel A shows a low power scan of the entire biopsy sample. The white box indicated the region magnified in Panel B. Panel B shows a spectrally unmixed high-power image of the same biopsy sample. CD8 appears as magenta; FoxP3 appears as red; PD-L1 appears as green; PD-1, melanoma cells, and CD4 are not shown.

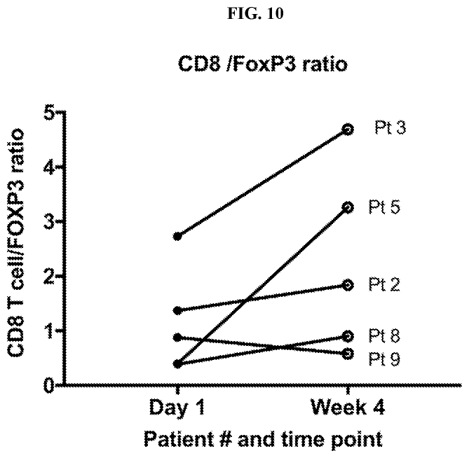

FIG. 10 shows a line graph of mIF results for melanoma patients 2, 3, 5, 8, and 9 demonstrating that treatment with X4P-001 increased the percentage of CD8⁺ cells in the tumor microenvironment (TME) relative to T_{reg} cells (FoxP3⁺).

### Proliferating T Cell Density in TME Post X4P-001 Treatment

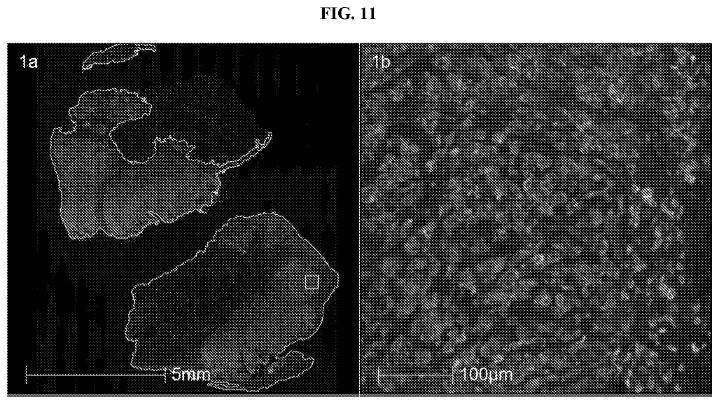

Biopsy samples from Day 1, Week 4, and End of Treatment (EOT) were stained with three (3) antibodies to detect CD8, melanoma cells, and proliferating cells. DAPI was used as nuclear counter stain. Representative CD8, Ki67, and melanoma cell staining from a pre-dose biopsy from patient 5 is shown in FIG. 11, Panels 1a and 1b. Panel 1a shows a low power scan of the entire biopsy sample. Panel 1b shows a spectrally unmixed high-power image of the invasive front. CD8 appears as green; melanoma cells appear as yellow; Ki67 appears as blue.

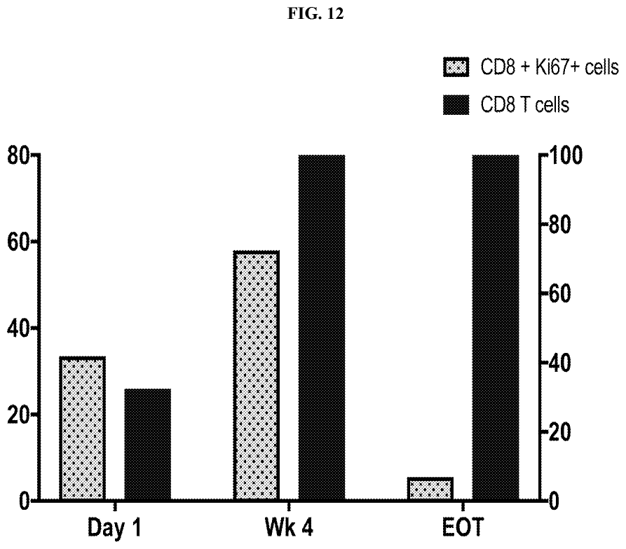

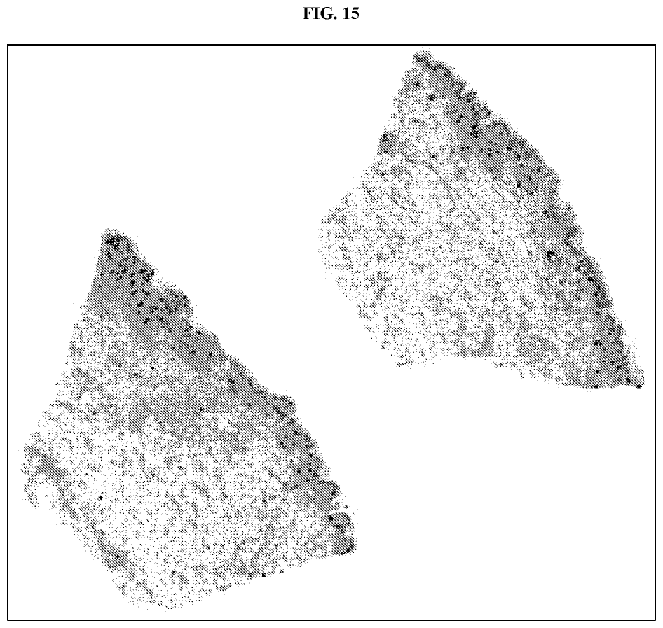

FIG. 12 shows a bar graph for CD8⁺ T cell density and proliferating CD8⁺ T cell (Ki67⁺) density across the entire tissue samples from patient 5. Monotherapy increased the densities of both cell populations with a stronger impact on proliferating T cells. The lack of CD8⁺Ki67⁺ T cells at the end of treatment is consistent with no residual tumor mass present in patient 5 following treatment (see FIG. 15).

CD8⁺ T Cell Infiltration into Melanoma Lesions

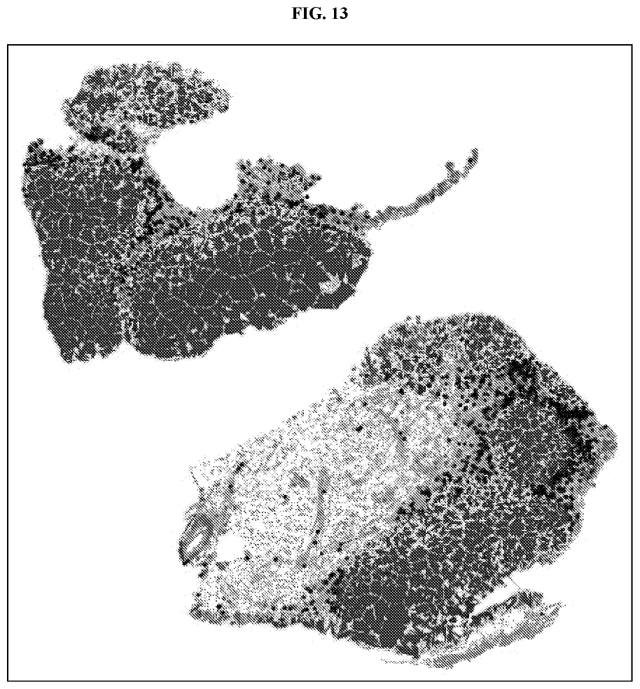

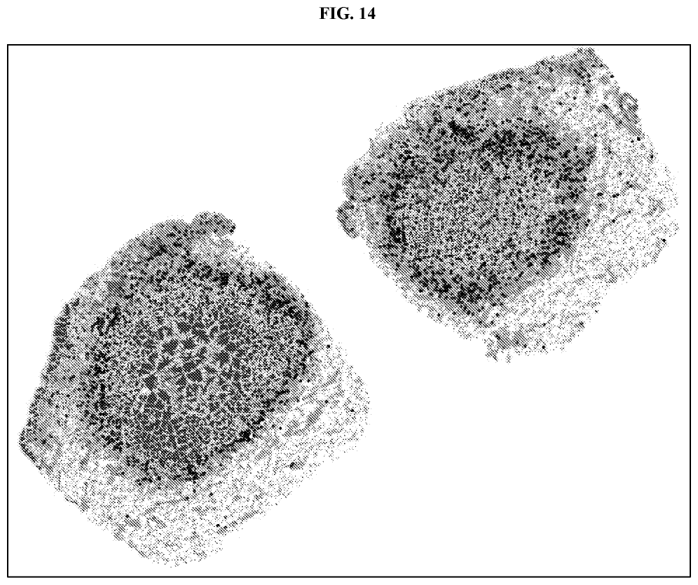

Representative distance measurements between CD8⁺ T cells and their nearest melanoma cell neighbors are shown in FIG. 13 (Day 1), FIG. 14 (Week 4), and FIG. 15 (End of treatment). Whole slide scans were performed using a fluorescence slide scanner (Aperio-FL, 20× objective). Images were imported into HALO for digital image analysis. The images represent the graphical output from the nearest neighbor analysis module, calculating the nearest CD8-to-tumor cell (blue line), CD8 (green), melanoma (yellow), Ki67 (red), Ki67⁺CD8⁺ T cells (black).

The average distance between CD8⁺ T cells and the nearest tumor cell on Day 1 was 95 microns. This distance decreased to 43 microns after 4 weeks of X4P-001 monotherapy. Further, the number of unique neighbors increased from 3,826 to 5,239, indicating enhanced CD8⁺ T cell infiltration. There was no residual tumor at the end of dual X4P-001/pembrolizumab treatment (FIG. 15).

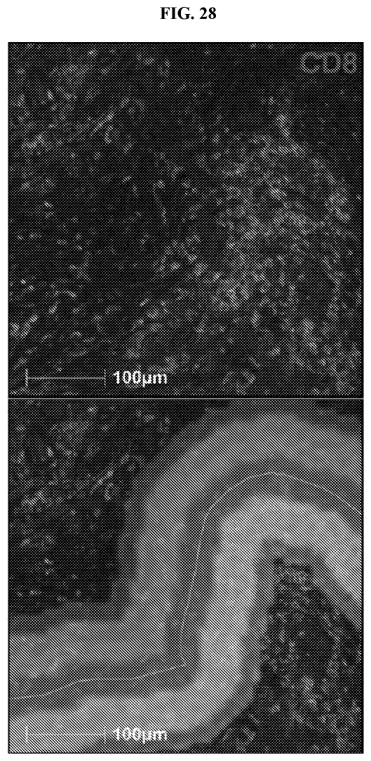

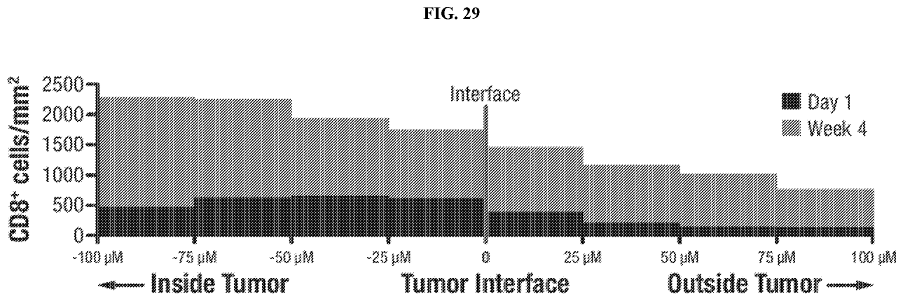

FIG. 28 and FIG. 29 show multiplex IHC and HALO image data demonstrating that X4P-001 monotherapy increases CD8⁺ cell density at the tumor interface in melanoma patients. CD8-labeled cells within 100 μM of the inside or outside of the tumor boundary with normal tissue were counted. The number of CD8⁺ cells/mm² was plotted against distance from the boundary in 25 μM bands. After 3 weeks of X4P-001 monotherapy, the total density of CD8⁺ cells within the boundary area was increased four-fold compared with baseline.

TABLE 5

| | | | CD8 Avg. | Total | |
|---|---|---|---|---|---|
| | | CD8 Within | Distance to | Interface | CD8 Avg. |
| Time- | CD8 | Interface | Interface | Area | Density |
| point | Count | Area | (μM) | (mm²) | (cells/mm²) |
| Day 1 | 8924 | 5233 | −21.21 | 13.7694 | 380.0449 |
| Week 4 | 25894 | 7557 | −18.36 | 4.8738 | 1550.5403 |

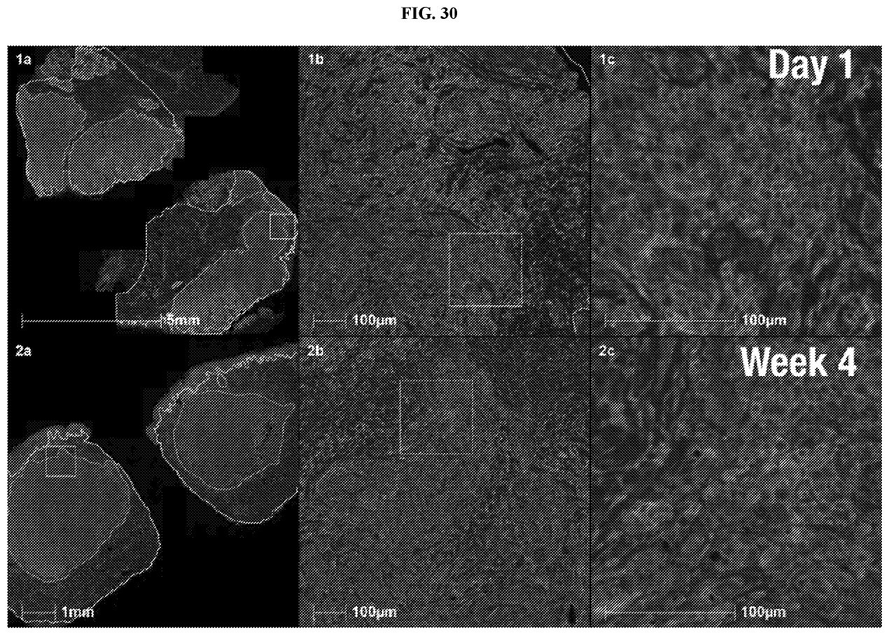

FIG. 30 shows IHC data demonstrating immune cell alterations at the tumor-normal cell interface following combination treatment (X4P-001 with pembrolizumab). Biopsy samples were obtained at baseline (top row) and at the end of X4P-001 monotherapy (bottom row). The left column shows biopsy samples with outlines of normal tissue (outer line) and the tumor border (inner line). The center column shows the enlarged boxed regions from the left column stained with the markers CD163, CD206, VISTA, COX-2, CD3, B7H3, and DAPI. The right column contains higher magnification views of the boxed regions in the center panel. X4P-001 leads to increased numbers of CD3⁺ cells within tumor borders and decreased expression of VISTA, a check point molecule that inhibits T cell activation and proliferation.

TABLE 6

| | | Cells/ | CD3/ | COX-2/ | CD206/ | VISTA/ | B7H3/ | CD163/ |
|---|---|---|---|---|---|---|---|---|
| Timepoint | ROI | mm² | mm² | mm² | mm² | mm² | mm² | mm² |
| Day 1 | Whole | 4149.31 | 308.53 | 2.87 | 259.38 | 1673.17 | 2998.14 | 464.86 |
| | Tissue | | | | | | | |
| | Tumor | 5758.23 | 239.91 | 3.40 | 264.79 | 2640.08 | 4826.36 | 426.51 |
| | Non- | 2057.63 | 400.31 | 2.07 | 252.20 | 410.11 | 615.63 | 514.86 |
| | Tumor | | | | | | | |
| Week 4 | Whole | 3572.93 | 738.82 | 1.97 | 182.26 | 457.53 | 2288.84 | 552.27 |
| | Tissue | | | | | | | |
| | Tumor | 5297.64 | 1050.68 | 2.98 | 161.25 | 852.86 | 3952.27 | 661.21 |
| | Non- | 2217.73 | 494.97 | 1.22 | 199.30 | 144.72 | 980.49 | 467.25 |
| | Tumor | | | | | | | |

### Biomarker Investigation Using Nanostring

For each gene biomarker, RNA was extracted from FFPE slides using Qiagen's AllPrep kit (Cat. 80234) and analyzed using NanoString nCounter platform with the PanCancer Immune probe set. Raw counts were normalized using the geometric mean of housekeeping genes, as described above.

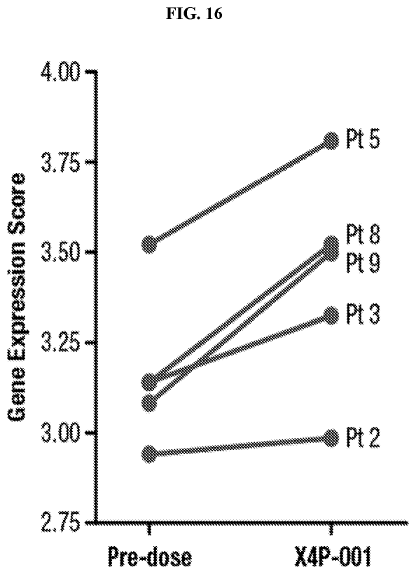

Antigen Presentation/Processing Gene Signature was calculated by taking the geometric mean of the normalized counts for eighteen (18) genes (B2M, CD74, CTSL, CTSS, HLA-DMA, HLA-DMB, HLA-DOB, HLA-DPA1, HLA-DPB1, HLA-DQA1, HLA-DQB1, HLA-DRA, HLA-DRB1, HLA-DRB3, PSMB8, PSMB9, TAP1, and TAP2) and then Log 10-transforming the mean to generate the Gene Expression Score. The pre-treatment and post-X4P-001 Log 10 transformed geometric gene count means for patients 2, 3, 5, 8, and 9 are summarized in Table 7. The Gene Expression Score increased for each patient from pre- and post-dosing of X4P-001, and is summarized in FIG. 16.

TABLE 7
Patient Antigen Presentation/Processing Gene Expression Scores

| Patient | Pre-Dose Score | Post-X4P-001 Score |
|---|---|---|
| 2 | 2.94 | 2.986 |
| 3 | 3.139 | 3.324 |
| 5 | 3.519 | 3.806 |
| 8 | 3.134 | 3.518 |
| 9 | 3.082 | 3.495 |

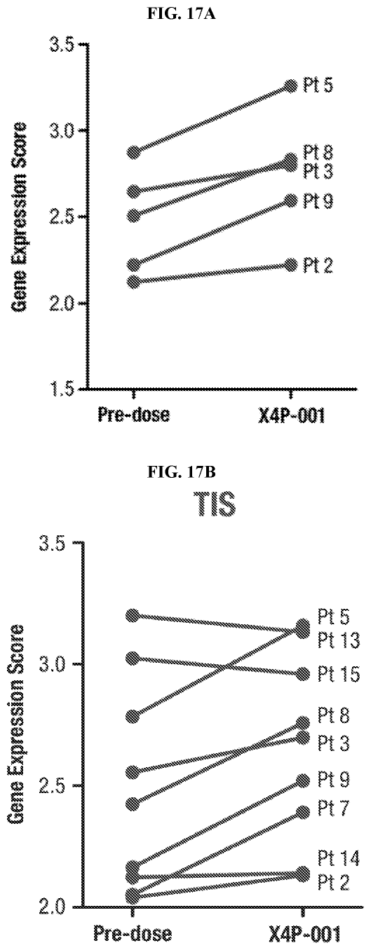

Tumor Inflammation Signature was calculated by taking the geometric mean of the normalized counts for eighteen (18) genes (CCL5, CD27, CD274, CD276, CD8A, CMKLR1, CXCL9, CXCR6, HLA-DQA1, HLA-DRB1, HLA-E, IDO1, LAG3, NKG7, PDCD1LG2, PSMB10, STAT1, and TIGIT) and then Log 10-transforming the mean to generate the Gene Expression Score. The pre-treatment and post-X4P-001 Log 10 transformed geometric gene count means for patients 2, 3, 5, 8, and 9 are summarized in Table 8. The Gene Expression Score increased for each patient from pre- and post-dosing of X4P-001, and is summarized in FIG. 17.

TABLE 8
Patient Tumor Inflammation Signature Gene Expression Scores

| Patient | Pre-Dose Score | Post-X4P-001 Score |
|---|---|---|
| 2 | 2.123 | 2.225 |
| 3 | 2.647 | 2.798 |
| 5 | 2.871 | 3.257 |
| 8 | 2.508 | 2.834 |
| 9 | 2.233 | 2.597 |

### Example 7—B16-OVA Syngeneic Melanoma Studies

### Impact of X4P-001 Mono- and Combination Therapies on Tumor Size

B16-OVA cells (˜1×10⁵) were implanted in C57BL/6 mice. Animals were evaluated periodically and when tumors attained a size of approximately 3 mm×3 mm, animals were then grouped randomly and treated for sixteen (16) days. Animals Treatments used are summarized in Table 9.

TABLE 9
Treatment Regiments in Syngeneic Tumor Models

| Group | X4-136 | αPD-L1 | αPD-1 | αCTCL-4 |
|---|---|---|---|---|
| Control | — | — | — | — |
| X4-136 | 100 mg/kg; 5 | — | — | — |
| | days on, 2 | | | |
| | days off | | | |
| αPD-L1 | — | 100 μg/ | — | — |
| | | mouse, every | | |
| | | other day | | |
| αPD-L1 + | 100 mg/kg; 5 | 100 μg/ | — | — |
| X4-136 | days on, 2 | mouse, every | | |
| | days off | other day | | |
| αPD-1 | — | — | 100 μg/ | — |
| | | | mouse, every | |
| | | | other day | |
| αPD-1 + | 100 mg/kg; 5 | — | 100 μg/ | — |
| X4-136 | days on, 2 | | mouse, every | |
| | days off | | other day | |
| αPD-L1 + | — | 100 μg/ | — | 100 μg/ |
| αCTCL-4 | | mouse, every | | mouse, every |
| | | other day | | fourth day |
| αPD-L1 + | 150 mg/kg; 5 | 100 μg/ | — | 100 μg/ |
| αCTCL-4 + | days on, 2 | mouse, every | | mouse, every |
| X4-136 | days off | other day | | fourth day |

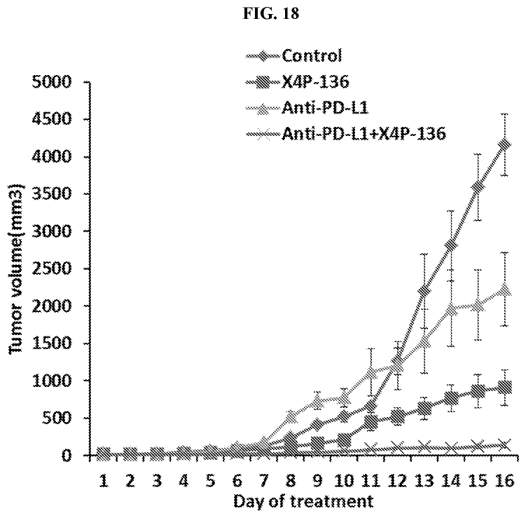

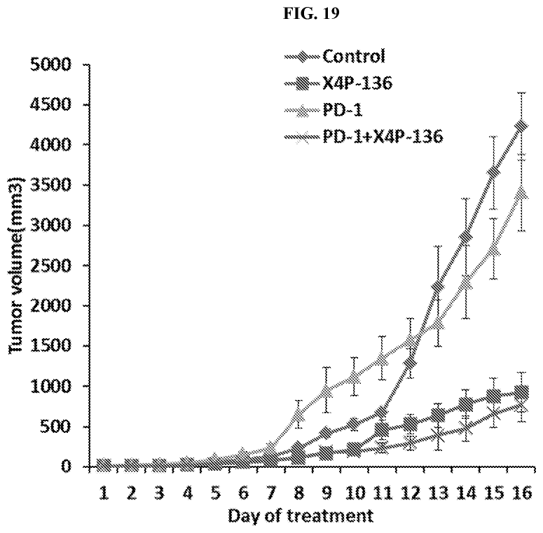

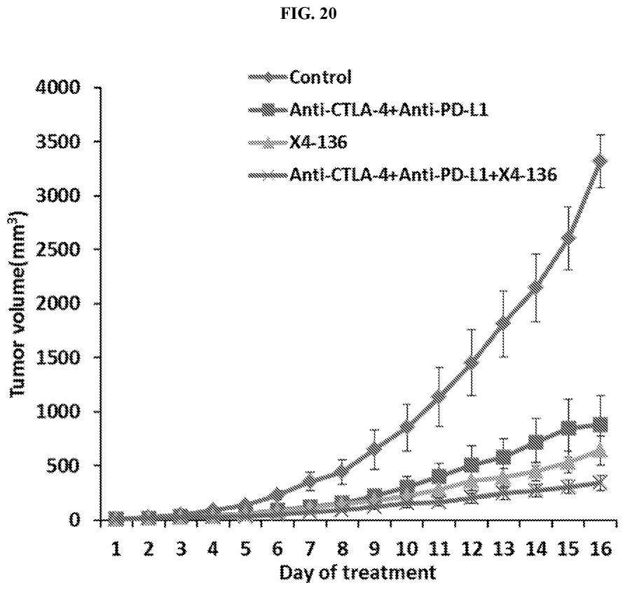

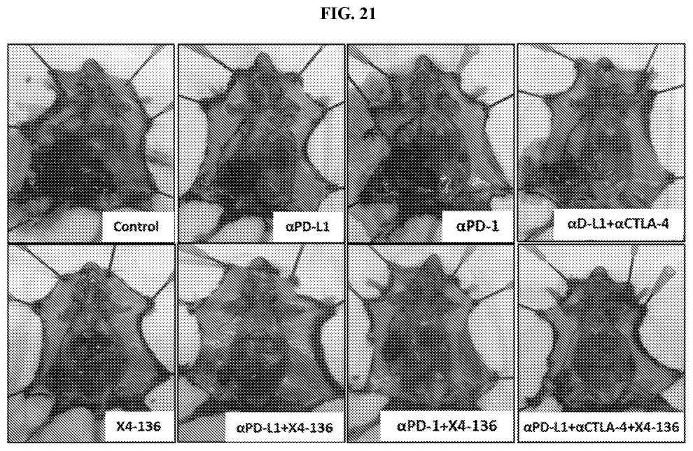

At the end of treatment animals were sacrificed and dissected to evaluate and sample tumor masts. Changes in tumor volume are summarized in FIGS. 18, 19, and 20. Depictions of tumor dissections are provided in FIG. 21. While X4P substantially reduced tumor volumes over the course of the study, combination with anti-PD-1, anti-PF-L1, and anti-CTCL-4+anti-PD-L1 greatly enhanced reduction in tumor volumes.

### Peripheral White Blood Cell Counts

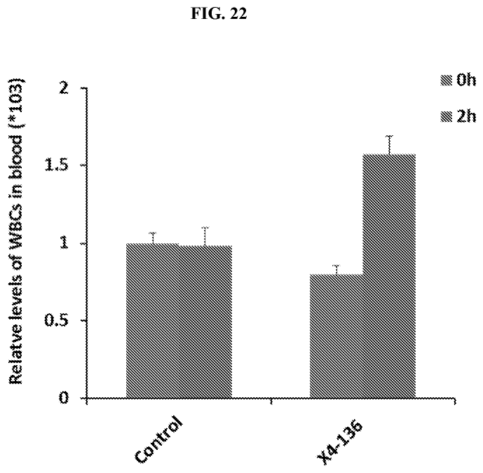

Serum samples from C57BL/6 mice with implanted B16-OVA tumors were collected prior to treatment and peripheral white blood cells were counted. Mice were then injected with vehicle or 100 mg/kg of X4P-001 and a second serum samples was collected two hours post injection and white blood cells were again counted. The results are summarized in FIG. 22. X4P-001 increased the number of peripheral white blood cells relative to the control.

### Modulation of Immune-Phenotype in TME

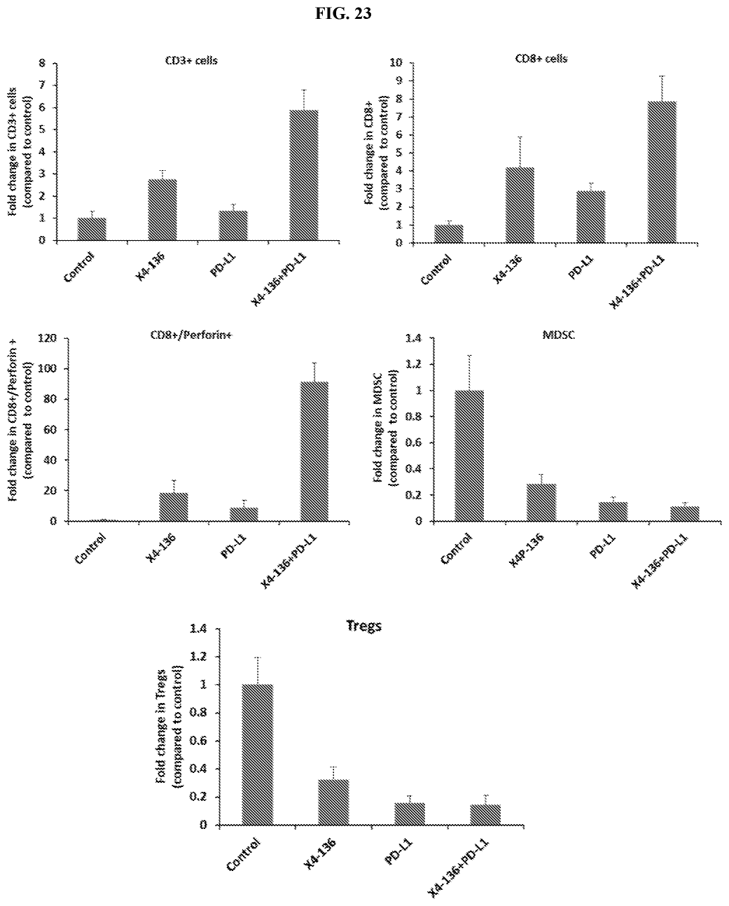

Single cell suspensions were prepared from tumor tissues by treating with collagenase and analyzing for various immune cell populations using flow cytometry. Cell surface markers included CD3, CD8, Perforin, CD15CD11b (MDSC), and FoxP3 (T_{reg}). Changes in TME immune cell phenotype are summarized in FIG. 23. X4P-001 increased the overall number of lymphocytes and CD8⁺ T cells in the TME relative to control. The enhancement was even greater for combination therapy with anti-PD-1. Importantly, monotherapy with X4P-001 or combination therapy with anti-PD-1 did not result in an increase in suppressor cells (T_{regs} and MDSC), but substantially decreased suppressor cell counts.

Western Blot Analysis of Tumor Cells Treated with Mono- or Combination Therapies

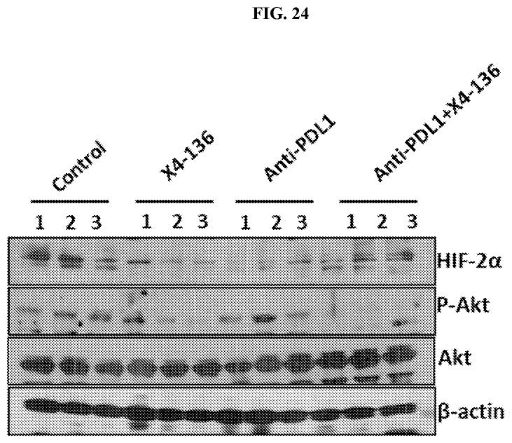

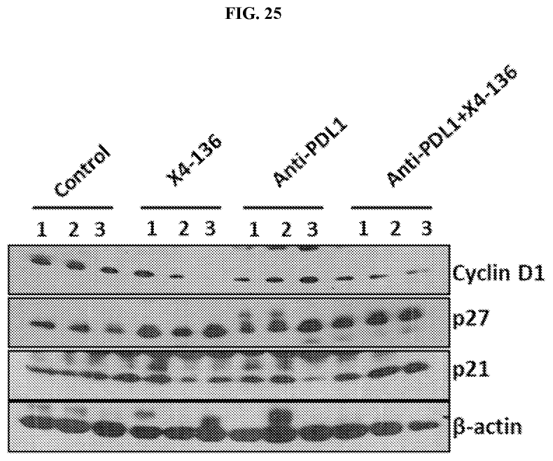

Tumor tissues were collected, flash frozen in liquid N₂ and lysed according to known methods. Protein quantities were normalized (e.g., BCA assay) and separated by gel electrophoresis. Proteins were then transferred to membranes for blotting. The results for HIF-2a expression and Akt activation are summarized in FIG. 24. The results for induction of p21 and p27, and the reduction of Cyclin D1 expression are summarized in FIG. 25.

### Example 9—In Vitro Mechanistic Experiments

### Transcriptional Activation via HIF-2α Response Elements and Inhibition of Invasion/Migration

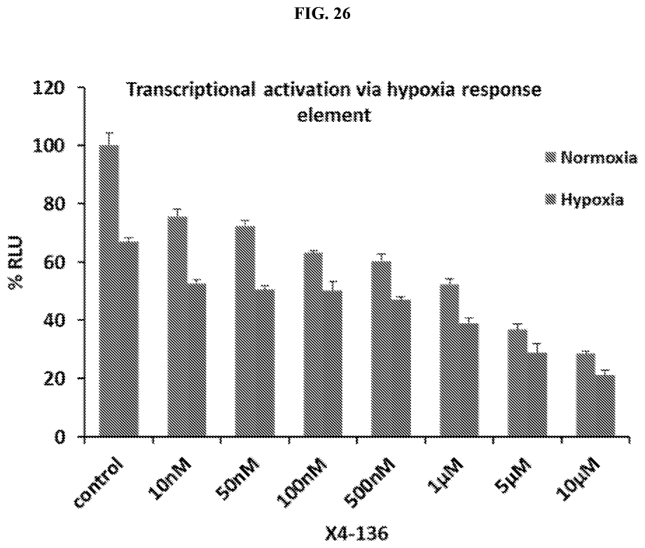

B16-OVA cells in normoxic and hypoxic conditions were transiently transfected with pHRE-luc and pRL-luc. Transfected cells were then incubated with different concentrations of X4P-001 ranging from 10 nM to 10 μM, or control. Luciferase activity was measured for cells in each condition using a dual luciferase assay kit. The results of the luciferase assay are summarized in FIG. 26.

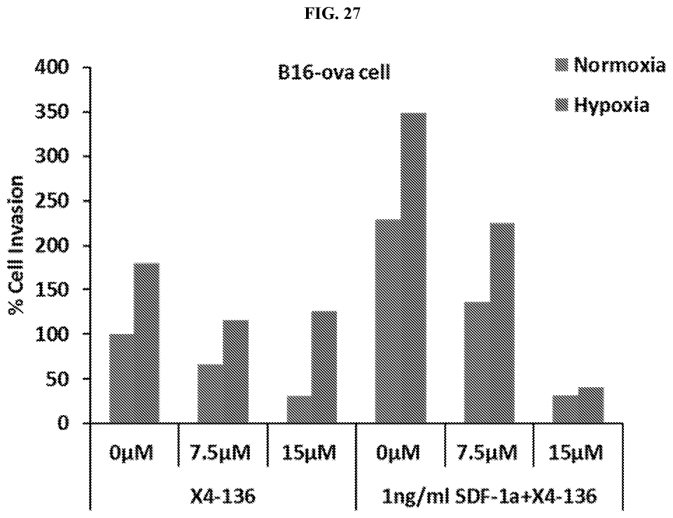

Transwell matrigel invasion chambers were used to assess the effect of X4P-001 on B16-OVA cell invasion. The Matrigel inserts and companion plates were prepared according to the manufacturer's instructions. B16-OVA cells were added to the chambers with X4P-001 (0 μM, 7.5 μM, or 15 μM) with or without 1 ng/mL SDF-1a. Matrigel Invasion chambers were incubated for 22 hours at 370° C., 5% CO₂ atmosphere. The non-invading cells were then scrubbed from the upper surface. The cells on the lower surface were fixed and stained, and cells counted. The percent invasion was calculated by determining the ratio of invading cells between the matrigel insert membrane and the control insert membrane. The results of the cell invasion assay are summarized in FIG. 27.

### REFERENCES

1. Ratajczak, et al. The pleotropic effects of the SDF-1-CXCR4 axis in organogenesis, regeneration, and tumorigenesis. Leukemia 2006:20; 1915-1924.
2. Scala, et al. Expression of CXCR4 predicts poor prognosis in patients with malignant melanoma. Clin Cancer Res 2005:11; 1835-1841.
3. Toyozawa, et al. Chemokine receptor CXCR4 is a novel marker for the progression of cutaneous malignant melanoma. Acta Histochem Cytochem. 2012; 45:293-299.
4. Kim, et al. CXCR4 signaling regulates metastasis of chemoresistant melanoma cells by a lymphatic metastatic niche. Cancer Res. 2010; 70:10411-10421.
5. Mosi R M, Anastassova V, Cox J, et al. The molecular pharmacology of AMD11070: An orally bioavailable CXCR4 HIV entry inhibitor. Biochem Pharmacol. 2012; 83:472-479.
6. D'Alterio, et al. Inhibition of stromal CXCR4 impairs development of lung metastases. Cancer Immunol Immunother. 2012:61; 1713-1720.
7. Feig, et al. Targeting CXCL12 from FAP-expressing carcinoma-associated fibroblasts synergizes with anti-PD-L1 immunotherapy in pancreatic cancer. PNAS 2013; 110:20212-20217.
8. Zhang et al. Preferential involvement of CXCR4 and CXCL12 in T cell migration toward melanoma cells. Cancer Biol Ther. 2006; 5:1034-1312.
9. Stone, et al. Multiple-Dose Escalation Study of the Safety, Pharmacokinetics, and Biologic Activity of Oral AMD070, a Selective CXCR4 Receptor Inhibitor, in Human Subjects. Antimicrob Agents Chemother. 2007; 51(7):2351-2358.
10. Moyle, et al. Proof of Activity with AMD11070, an Orally Bioavailable Inhibitor of CXCR4-Tropic HIV Type 1. Clin Infect Dis. 2009; 48:798-805.
11. Robert, et al. Pembrolizumab versus Ipilimumab in Advanced Melanoma. N Engl J Med. 2015; 372:2521-2532.
12. Tumeh, et al. PD-1 blockade induces responses by inhibiting adaptive immune resistance. Nature 2014:515; 568-571.
13. Tarhini, et al. Immune Monitoring of the Circulation and the Tumor Microenvironment in Patients with Regionally Advanced Melanoma Receiving Neoadjuvant Ipilimumab. PLoS One 2014; 9(2):e87705.
14. Nyunt, et al. Pharmacokinetic Effect of AMD070, an Oral CXCR4 Antagonist, on CYP3A4 and CYP2D6 Substrates Midazolam and Dextromethorphan in Healthy Volunteers. J Acquir Immune Defic Syndr. 2008; 47:559-565.
15. Cao et al. Effect of Low-Dose Ritonavir on the Pharmacokinetics of the CXCR4 Antagonist AMD070 in Healthy Volunteers. Antimicrob Agents Chemother. 2008; 52:1630-1634.
16. Common Terminology Criteria for Adverse Events (CTCAE). Version 4.0, 28 May 2009. U.S. Department of Health and Human Services, National Institutes of Health, National Cancer Institute. NIH Publication No. 03-5410.
17. NCI CTCAE v4.03, 14 Jun. 2010 available at (accessed 6 Apr. 2015): http://evs.nci.nih.gov/ftpl/CTCAE/CTCAE_4.03_2010-06-14_QuickReference_5x7.pdf
18. WMA Declaration of Helsinki—Ethical Principles for Medical Research Involving Human Subjects. Available at (accessed 6 Apr. 2015) http://www.wma.net/en/30publications/10policies/b3/
19. Vanharanta et al. Epigenetic expansion of VHL-HIF signal output drives multiorgan metastasis in renal cancer. Nat Med 2013; 19: 50-6.
20. Gale and McColl, Chemokines: extracellular messengers for all occasions? BioEssays 1999; 21: 17-28.
21. Highfill et al., Disruption of CXCR2-mediated MDSC tumor trafficking enhances anti-PD1 efficacy. Sci Transl Med 2014; 6: ra67.
22. Facciabene et al., Tumour hypoxia promotes tolerance and angiogenesis via CCL28 and Treg cells. Nature 2011; 475: 226-230.
23. Montane et al., Prevention of murine autoimmune diabetes by CCL22-mediated Treg recruitment to pancreatic islets. J Clin Invest 2011; 121: 3024-8.
24. Acharyya et al., CXCL1 paracrine network links cancer chemoresistance and metastasis. Cell 2012; 150: 165-78.
25. Zhao et al., TNF signaling drives myeloid-derived suppressor cell accumulation. J Clin Invest 2012; 122: 4094-4104.
26. Silva et al., Profiling essential genes in human mammary cells by multiplex RNA1 screening. Science 2008; 319: 617-20.
27. Schlabach et al., Cancer proliferation gene discovery through functional genomics. Science 2008; 319: 620-24.
28. Shen et al., CXCR4-mediated STAT3 activation is essential for CXCL12-induced invasion in bladder cancer. Tumour Biol 2013; 34: 1839-45.
29. Ayers et al., IFN-γ-related mRNA profile predicts clinical response to PD-1 blockade. Journal of Clinical Investigation 2017, 127(8), 2930-2940.
30. Sharma, P. et al., Primary, Adaptive, and Acquired Resistance to Cancer Immunotherapy. Cell 2017, 168, 707-723.

We claim:

1. A method of identifying a patient with a cancerous tumor who will benefit from treatment with a CXCR4 inhibitor, comprising:
(a) obtaining a first tumor sample prior to administration of the CXCR4 inhibitor to the patient;
(b) measuring a level in the first tumor sample of one or more biomarkers selected from CD8⁺ T cells or CD8⁺ T cells/T_{reg} ratio, CD8⁺Ki-67⁺ T cells, granzyme B, an IFN-γ signature score, a CTL signature score, an antigen presentation/processing signature score, a tumor inflammation signature score, a VISTA biomarker panel, or PD-L1 expression;
(c) administering to the patient an effective amount of a CXCR4 inhibitor and optionally an immunotherapeutic agent;
(d) obtaining a second tumor sample after administration of the CXCR4 inhibitor to the patient; and
(e) measuring a level in the second tumor sample of one or more biomarkers selected from CD8⁺ T cells or CD8⁺ T cells/T_{reg} ratio, CD8⁺Ki-67⁺ T cells, granzyme B, an IFN-γ signature score, a CTL signature score, an antigen presentation/processing signature score, a tumor inflammation signature score, a VISTA biomarker panel, or PD-L1 expression.
wherein the tumor response to step (c) is predictive of the likelihood of successful treatment of the tumor based on a greater or lesser response of the tumor compared with one or more similar patients and as evaluated using one or more of the biomarkers.

2. A method of predicting a patient response to a CXCR4 inhibitor optionally in combination with an immunotherapeutic agent, comprising the steps of:
(a) obtaining a first tumor sample prior to administration of the CXCR4 inhibitor to the patient;
(b) measuring a level in the first tumor sample of one or more biomarkers selected from CD8⁺ T cells or CD8⁺ T cells/T_{reg} ratio, CD8⁺Ki-67⁺ T cells, granzyme B, an IFN-γ signature score, a CTL signature score, an antigen presentation/processing signature score, a tumor inflammation signature score, a VISTA biomarker panel, or PD-L1 expression;
(c) administering to the patient an effective amount of a CXCR4 inhibitor and optionally an immunotherapeutic agent;
(d) obtaining a second tumor sample after administration of the CXCR4 inhibitor to the patient; and
(e) measuring a level in the second tumor sample of one or more biomarkers selected from CD8⁺ T cells or CD8⁺ T cells/T_{reg} ratio, CD8⁺Ki-67⁺ T cells, granzyme B, an IFN-γ signature score, a CTL signature score, an antigen presentation/processing signature score, a tumor inflammation signature score, a VISTA biomarker panel, or PD-L1 expression;
wherein the tumor response to step (c) is predictive of the likelihood of successful treatment of the tumor based on a greater or lesser response of the tumor compared with one or more similar patients and as evaluated using one or more of the biomarkers.

3. The method of claim 1, wherein the CXCR4 inhibitor is selected from X4P-001 or X4-136, or a pharmaceutically acceptable salt thereof.

4. The method of claim 1, wherein the immunotherapeutic agent is an immune checkpoint inhibitor.

5. The method of claim 4, wherein the immune checkpoint inhibitor is selected from ipilimumab (Yervoy®), atezolizumab (Tecentriq®), nivolumab (Opdivo®), pidilizumab, avelumab (Bavencio®), durvalumab (Imfinzi®), PDR001, REGN2810, or pembrolizumab (Keytruda®).

6. The method of claim 5, wherein the immune checkpoint inhibitor is pembrolizumab.

7. The method of claim 1, wherein the IFN-7 signature score comprises an increase in one or more of IDO1, CXCL10, CXCL9, HLA-DRA, STAT1 and IFN-γ.

8. The method of claim 1, wherein the CTL signature score comprises an increase in one or more of CD8A, CD8B, FLTLG, GZMM, or PRF1.

9. The method of claim 1, wherein the antigen presentation/processing gene score comprises an increase in one or more of B2M, CD74, CTSL, CTSS, HLA-DMA, HLA-DMB, HLA-DOB, HLA-DPA1, HLA-DPB1, HLA-DQA1, HLA-DQB1, HLA-DRA, HLA-DRB1, HLA-DRB3, PSMB8, PSMB9, TAP1, or TAP2.

10. The method of claim 1, wherein the tumor inflammation gene score comprises an increase in one or more of CCL5, CD27, CD274, CD276, CD8A, CMKLR1, CXCL9, CXCR6, HLA-DQA1, HLA-DRB1, HLA-E, IDO1, LAG3, NKG7, PDCD1LG2, PSMB10, STAT1, or TIGIT.

11. The method of claim 1, wherein the cancer is selected from renal cell cancer, melanoma, liver cancer, hepatocellular carcinoma, hepatocholangiocarcinoma, ovarian cancer, ovarian epithelial cancer, fallopian tube cancer, papillary serous cystadenocarcinoma, uterine papillary serous carcinoma (UPSC); prostate cancer; testicular cancer, gall bladder cancer, adrenocortical adenocarcinoma, colon cancer, pancreatic cancer, pancreatic carcinoma, brain cancer, gastrointestinal/stomach (GIST) cancer, medulloblastoma, glioma, glioblastoma, squamous cell carcinoma of the head and neck (SCCHN), Waldenstrom's macroglobulinemia, breast cancer, urothelial carcinoma, head and neck cancer, and cervical cancer.

12. The method of claim 11, wherein the cancer is advanced or metastatic melanoma.

13. The method of claim 11, wherein the melanoma is unresectable advanced or unresectable metastatic melanoma.

14. A method of predicting a patient response to a checkpoint inhibitor after treatment with a CXCR4 inhibitor, comprising the steps of:
(a) obtaining a first tumor sample from the patient prior to administration of the CXCR4 inhibitor to the patient;
(b) measuring a level in the first tumor sample of one or more biomarkers selected from CD8⁺ T cells or CD8⁺ T cells/T_{reg} ratio, CD8⁺Ki-67⁺ T cells, granzyme B, an IFN-γ signature score, a CTL signature score, an antigen presentation/processing signature score, a tumor inflammation signature score, a VISTA biomarker panel, or PD-L1 expression;
(c) administering to the patient an effective amount of a CXCR4 inhibitor and optionally an immunotherapeutic agent;
(d) obtaining a second tumor sample after administration of the CXCR4 inhibitor to the patient; and
(e) measuring a level in the second tumor sample of one or more biomarkers selected from CD8⁺ T cells or CD8⁺ T cells/T_{reg} ratio, CD8⁺Ki-67⁺ T cells, granzyme B, an IFN-γ signature score, a CTL signature score, an antigen presentation/processing signature score, a tumor inflammation signature score, a VISTA biomarker panel, or PD-L1 expression;
wherein the tumor response to step (c) is predictive of the likelihood of successful treatment of the tumor with a checkpoint inhibitor after treatment with a CXCR4 inhibitor, based on a greater or lesser response of the tumor compared with one or more similar patients and as evaluated using one or more of the biomarkers.

15. The method of claim 14, wherein the patient initially does not respond to treatment with a checkpoint inhibitor.

16. The method of claim 14, wherein the patient initially responds to treatment with a checkpoint inhibitor, but has become refractory to treatment with the checkpoint inhibitor.

17. The method of claim 14, wherein the VISTA biomarker panel is selected from one or more of CD163, CD206, VISTA, COX-2, CD3, and B7H3 biomarkers.

18. The method of claim 17, wherein the VISTA biomarker panel is VISTA.

19. The method of claim 1, wherein the VISTA biomarker panel is selected from one or more of CD163, CD206, VISTA, COX-2, CD3, and B7H3 biomarkers.
